(19) United States
(12) Patent Application Publication
(10) Pub. No.: US 2024/0296904 A1
(43) Pub. Date: Sep. 5, 2024

(54) METHOD AND APPARATUS FOR PREDICTING RNA-PROTEIN INTERACTION, MEDIUM AND ELECTRONIC DEVICE

(71) Applicant: BOE Technology Group Co., Ltd., Beijing (CN)

(72) Inventor: Zhenzhong ZHANG, Beijing (CN)

(73) Assignee: BOE Technology Group Co., Ltd., Beijing (CN)

(21) Appl. No.: 18/025,394

(22) PCT Filed: Sep. 29, 2021
(86) PCT No.: PCT/CN2021/121878

§ 371 Date: Mar. 8, 2023

(51) Int. Cl. G16B 15/30; G16B 40/20

(52) U.S. Cl. CPC G16B 15/30; G16B 40/20

(57) ABSTRACT

The present disclosure provides a method and apparatus for predicting an RNA-protein interaction, a medium and an electronic device, relating to the field of artificial intelligence technologies. The method includes: acquiring a candidate RNA sequence and a candidate protein sequence; encoding the candidate RNA sequence to obtain an RNA vector sequence; encoding the candidate protein sequence to obtain a protein vector sequence; constructing a matching feature matrix according to the RNA vector sequence and the protein vector sequence; performing feature extraction on the matching feature matrix, and determining the interaction between the candidate RNA sequence and the candidate protein sequence according to the extracted matching feature.

### CROSS-REFERENCE TO RELATED APPLICATIONS

The present application is the U.S. National phase application of International Application No. PCT/CN2021/121878, filed on Sep. 29, 2021, the entire contents of which are hereby incorporated by reference in its entirety.

### TECHNICAL FIELD

The present disclosure relates to the field of artificial intelligence technologies, and in particular, to a method for predicting an RNA-protein interaction, an apparatus for predicting an RNA-protein interaction, a computer-readable storage medium and an electronic device.

### BACKGROUND

Noncoding RNA (ncRNA) is involved in many complex processes of cellular activity, plays an important role in the life processes such as alternative splicing, chromatin modification and epigenetics processes, and is closely related to many diseases. Researches have indicated that most noncoding RNAs achieve their regulatory function by interacting with proteins. Therefore, the research on the interaction between noncoding RNA and protein has important significance for disclosing the molecular action mechanism of noncoding RNA in human diseases and life activities, and becomes one of the important ways for analyzing the functions of noncoding RNA and protein at present.

It should be noted that the information disclosed in the Background section above is only for enhancement of understanding of the background of the present disclosure, and thus may include information that does not constitute prior art known to those of ordinary skill in the art.

### SUMMARY

The present disclosure provides a method for predicting an RNA-protein interaction, an apparatus for predicting an RNA-protein interaction, a computer-readable storage medium and an electronic device.

The present disclosure provides a method for predicting an RNA-protein interaction, including:
acquiring a candidate RNA sequence and a candidate protein sequence;
encoding the candidate RNA sequence to obtain an RNA vector sequence;
encoding the candidate protein sequence to obtain a protein vector sequence;
constructing a matching feature matrix according to the RNA vector sequence and the protein vector sequence; and
performing feature extraction on the matching feature matrix, and determining, according to an extracted matching feature, an interaction between the candidate RNA sequence and the candidate protein sequence.

The present disclosure provides a non-transitory computer-readable storage medium, having a computer program stored thereon, and the computer program, when performed by a processor, causes the processor to implement the above-described method.

The present disclosure provides an electronic device, including: a processor; and a memory for storing executable instructions for the processor; where the processor is configured to perform the above-described method by executing the executable instructions.

It should be understood that both the foregoing general description and the following detailed description are exemplary and explanatory only and not for limiting the present disclosure.

### BRIEF DESCRIPTION OF THE FIGURES

Accompanying figures herein, which are incorporated in the specification and constitute a part of the present specification, illustrate embodiments conforming to the present disclosure, and are used to explain the principles of the present disclosure together with the specification. It is apparent that the accompanying figures described below are only some of embodiments of the present disclosure, and other accompanying figures may also be obtained according to these accompanying figures without any creative efforts by those of ordinary skill in the art.

FIG. 1 illustrates a schematic diagram of exemplary system architecture applicable to a method and apparatus for predicting an RNA-protein interaction according to the embodiments of the present disclosure.

FIG. 2 schematically illustrates a flowchart of a method for predicting an RNA-protein interaction according to one embodiment of the present disclosure.

FIG. 3 schematically illustrates a flowchart for obtaining an RNA vector sequence according to one embodiment of the present disclosure.

FIG. 4 schematically illustrates a flowchart for obtaining a protein vector sequence according to another embodiment of the present disclosure.

FIG. 5 schematically illustrates a flowchart for constructing a matching feature matrix according to one embodiment of the present disclosure.

FIG. 6 schematically illustrates a flowchart for model training according to one embodiment of the present disclosure.

FIG. 7 schematically illustrates a flowchart of a method for predicting an RNA-protein interaction according to another embodiment of the present disclosure.

FIG. 8 schematically illustrates a block diagram of an apparatus for predicting an RNA-protein interaction according to one embodiment of the present disclosure.

FIG. 9 illustrates a schematic structural diagram of a computer system suitable for implementing an electronic device according to the embodiments of the present disclosure.

### DETAILED DESCRIPTION

Exemplary embodiments will now be described more fully with reference to the accompanying figures. However, the exemplary embodiments may be implemented in various forms and should not be construed as limited to the examples set forth herein; rather, these embodiments are provided so that the present disclosure will be more comprehensive and complete, and will fully convey the concept of the exemplary embodiments to those skilled in the art. The described features, structures, or characteristics may be combined in any suitable manner in one or more embodiments. In the following description, many specific details are provided so as to give a full understanding of the embodiments of the present disclosure. However, those skilled in the art will appreciate that one or more of specific details may be omitted in practicing the technical solutions of present disclosure, or other methods, components, devices, steps, etc. may be employed. In other instances, the well-known technical solutions will not be shown or described in detail so as to avoid obscuring various aspects of present disclosure through distraction.

In addition, the figures are merely schematic representations of the present disclosure and are not necessarily drawn to scale. The same reference numerals in the figures denote the same or similar parts, and the repeated description thereof will be omitted. Some of the block diagrams shown in the figures are functional entities and do not necessarily have to physically or logically correspond to separate entities. These functional entities may be implemented in software, or implemented in one or more hardware modules or integrated circuits, or implemented in different network and/or processor devices and/or microcontroller devices.

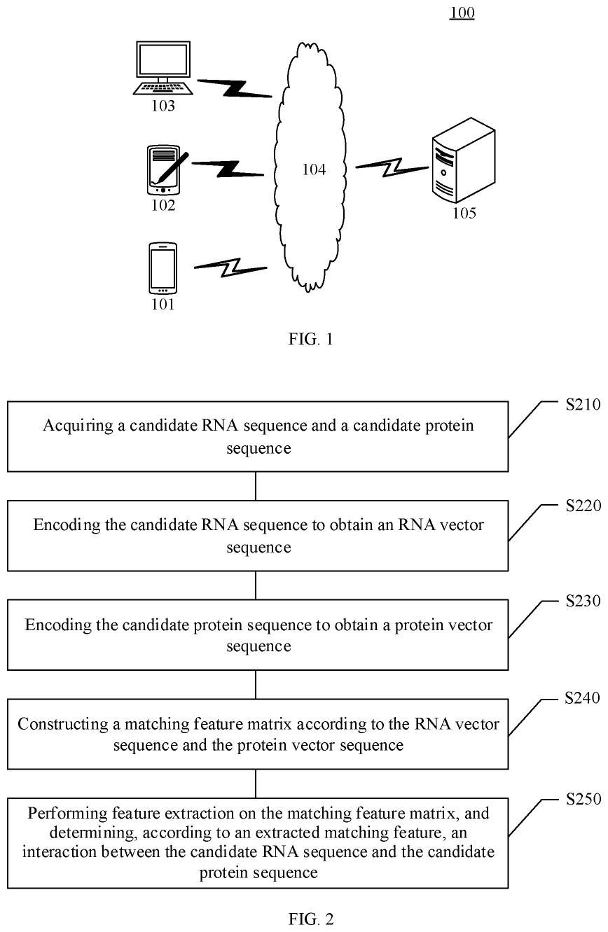

FIG. 1 illustrates a schematic diagram of system architecture for exemplary application environments applicable to a method and apparatus for predicting an RNA-protein interaction according to the embodiments of the present disclosure.

As shown in FIG. 1, the system architecture 100 of an interaction prediction system may include: one or more terminal devices among terminal devices 101, 102, 103, a network 104, and a server 105. The network 104 is the medium used to provide communication links between the terminal devices 101, 102, 103 and the server 105. The network 104 may include various connection types, for example, wired communication links, wireless communication links, or fiber optic cables and the like. The terminal devices 101, 102, 103 may be various electronic devices including, but not limited to, desktop computers, portable computers, smart phones, tablet computers and the like. It should be understood that the number of terminal devices, networks, and servers in FIG. 1 is merely illustrative. There may be any number of terminal devices, networks, and servers, as desired for an implementation. For example, the server 105 may be one server, a server cluster composed of a plurality of servers, a cloud computing platform, or a virtualization center. Specifically, the server 105 may be used to perform: acquiring a candidate RNA sequence and a candidate protein sequence; encoding the candidate RNA sequence to obtain an RNA vector sequence; encoding the candidate protein sequence to obtain a protein vector sequence; constructing a matching feature matrix according to the RNA vector sequence and the protein vector sequence; and performing feature extraction on the matching feature matrix, and determining the interaction between the candidate RNA sequence and the candidate protein sequence according to the extracted matching feature.

The method for predicting the RNA-protein interaction provided by the embodiments of the present disclosure is generally performed by the server 105. Accordingly, the apparatus for predicting the RNA-protein interaction is generally disposed in the server 105, and the server may send the prediction result of the interaction between the candidate RNA sequence and the candidate protein sequence to the terminal device, and the terminal device displays the prediction result to the user. It will be easily understood by those skilled in the art that the method for predicting the RNA-protein interaction provided by the embodiments of the present disclosure may also be performed by one or more of the terminal devices 101, 102, and 103. Accordingly, the apparatus for predicting the RNA-protein interaction may also be disposed in the terminal devices 101, 102, and 103, for example, after being performed by the terminal device, the prediction result may be directly displayed on a display screen of the terminal device, or the prediction result may be provided to the user by way of voice broadcast, which is not specifically limited in this exemplary embodiment.

The technical solutions of the embodiments of the present disclosure are explained in detail as follows.

This exemplary embodiment provides a method for predicting an RNA-protein interaction, which may be applied to both the above server 105 and one or more of the above terminal devices 101, 102, and 103, and is not specifically limited in this exemplary embodiment. Referring to FIG. 2, the method for predicting the RNA-protein interaction may include the following steps S210 to S250.

Step S210, acquiring a candidate RNA sequence and a candidate protein sequence.

Step S220, encoding the candidate RNA sequence to obtain an RNA vector sequence.

Step S230, encoding the candidate protein sequence to obtain a protein vector sequence.

Step S240, constructing a matching feature matrix according to the RNA vector sequence and the protein vector sequence.

Step S250, performing feature extraction on the matching feature matrix, and determining, according to an extracted matching feature, an interaction between the candidate RNA sequence and the candidate protein sequence.

In the method for predicting the RNA-protein interaction provided by this exemplary embodiment of the present disclosure, the candidate RNA sequence and the candidate protein sequence are acquired; the candidate RNA sequence is encoded to obtain the RNA vector sequence; the candidate protein sequence is encoded to obtain the protein vector sequence; the matching feature matrix is constructed according to the RNA vector sequence and the protein vector sequence; and the feature extraction is performed on the matching feature matrix, and the interaction between the candidate RNA sequence and the candidate protein sequence is determined according to the extracted matching feature. The present disclosure can improve the accuracy of RNA-protein interaction prediction by: constructing the matching feature matrix by using the matching relationship between the RNA sequence and the protein sequence, and predicting the interaction between the RNA sequence and the protein sequence according to the matching feature matrix.

The above steps of this exemplary embodiment will be described in more detail below.

In step S210, the candidate RNA sequence and the candidate protein sequence are acquired.

In this exemplary embodiment, at least one candidate RNA-protein pair, which consists of an RNA sequence and a protein sequence, may be acquired, and the interaction between the RNA sequence and the protein sequence in each candidate RNA-protein pair is unknown. For example, the user may input the candidate RNA-protein pair through the terminal device. For example, the user may input the candidate RNA-protein pair by manual input or by voice input, which is not specifically limited in this example. For example, an RNA sequence may be input, followed by a protein sequence, without any limitation on the order in which they are entered. For example, the RNA sequence and the protein sequence may be input into different text boxes, or may be input into the same text box. For example, after the input is completed, the “start prediction” button is clicked, and the prediction steps provided in some embodiments of the present application are started.

The interaction between RNA and protein means that the function of protein is expressed by interaction with other proteins and RNA. For example, the interaction of protein and RNA plays an important role in the synthesis of proteins. Meanwhile, many functions of RNA are also dependent on interactions with proteins. The interaction may be regulation, guidance, etc., which is not limited herein. For example, in the presence of an interaction, the RNA may direct the synthesis of proteins, or the RNA may regulate the functioning of proteins. The interaction between RNA and protein may also mean that the two may regulate each other's life cycles and functions through physical interaction. For example, an RNA encoding sequence may direct the synthesis of a protein, and correspondingly, a protein may also regulate the expression and function of RNA.

After acquiring the candidate RNA-protein pair, an interaction prediction system may be used to predict the interaction of each input candidate RNA-protein pair and determine whether each candidate RNA-protein pair has an interaction or not according to the prediction result. Meanwhile, the prediction result of the interaction of the candidate RNA-protein pair may be output to the terminal device for the user to view. For example, the prediction result may be directly displayed on a display screen of the terminal device, or the prediction result may be provided to the user by way of voice broadcast, which is not specifically limited in this example.

In other examples, at least one candidate RNA sequence may also be acquired, and the protein sequence that has an interaction with each input candidate RNA sequence may be searched in the database. For example, after the user inputs the candidate RNA sequence through the terminal device, the user may select at least one protein sequence in the database, and a plurality of RNA-protein pairs may be constituted by the candidate RNA sequence and respective protein sequences, and then the interaction of each RNA-protein pair may be predicted through the interaction prediction system, and a protein sequence that can interact with the candidate RNA sequence may be output according to the prediction result. Preferably, several kinds of protein sequences may be pre-stored in the database, so as to facilitate calling when predicting the interaction of the RNA-protein pair. For example, the protein sequence may be stored in a Redis database, or may be stored in a MySQL database, and in turn, the candidate protein sequence may be queried and selected in real time. The Redis is a key-value storage system, and when stored in the Redis database, the key-value storage system may include: a key-value pair formed by a sequence identifier (such as a sequence number) and a corresponding protein sequence, in which the key is the sequence identifier and the value is the corresponding protein sequence. Redis is an efficient caching technology and can support a read/write frequency over 100K+ per second, and it has certain advantages in terms of data reading and storing speeds. MySQL is an associative database management system, where an associative database stores data in different tables instead of uniformly storing all data, thereby increasing the storage speed and improving the flexibility, having stable advantages in terms of data storage and avoiding data loss.

It will be understood that several kinds of RNA sequences may also be pre-stored in the database for recall in predicting the interaction of RNA-protein pair. Therefore, at least one candidate protein sequence may also be acquired, and the RNA sequence that has an interaction with each input candidate protein sequence may be searched in the database. Similarly, after the user inputs the protein sequence through the terminal device, the user may select at least one RNA sequence in the database, a plurality of RNA-protein pairs may be constituted by the candidate protein sequence and respective RNA sequences, and then the interaction of each RNA-protein pair may be predicted through the interaction prediction system, and output an RNA sequence that can interact with the candidate protein sequence according to the prediction result, which is not specifically limited by the present disclosure.

In step S220, the candidate RNA sequence is encoded to obtain the RNA vector sequence.

After acquiring the candidate RNA sequence, the candidate RNA sequence may be encoded to obtain an RNA vector sequence, and a matching feature matrix will be constructed according to the RNA vector sequence and the protein vector sequence, so that the interaction between the RNA vector sequence and the protein vector sequence will be predicted based on the matching feature matrix.

In one exemplary embodiment of the present disclosure, the RNA sequence may be represented by a base sequence, for example, one RNA sequence may be represented as AGCAUAGCACCU . . . . For an RNA sequence, 4 bases may be included as follows: adenine (A), uracil (U), guanine (G), and cytosine (C). Correspondingly, an RNA sequence may also be represented using a base k-mer subsequence. K-mer subsequence refers to a k-linkages consisting of k bases or k types of amino acids. Specifically, 4 bases may be arranged to obtain all base k-mer subsequences, and for a certain k value, 4^{k} base k-mer subsequences may be obtained. For example, when k is equal to 3, there are a total of 4³=64 base 3-mer subsequences, and when k is equal to 4, there are a total of 4⁴=256 base 4-mer subsequences. For example, AGC, AUA, GCA and CCU are four different base 3-mer subsequences, and AGCA, UAGC and ACCU are three different base 4-mer subsequences. Thus, RNA sequence AGCAUAGCACCU . . . may also be denoted as {AGC, AUA, GCA, CCU, . . . }, and may also be denoted as {AGCA, UAGC, ACCU, . . . }. In other examples, the RNA sequence may be read in an overlapping manner to obtain corresponding base 3-mer subsequences or base 4-mer subsequences. Correspondingly, the base 3-mer subsequences of the RNA sequence may further include AGC, GCA, CAU, AUA, etc., and the base 4-mer subsequences of the RNA sequence may further include AGCA, GCAU, CAUA, etc., which are not specifically limited by the present disclosure. In an exemplary embodiment of the present disclosure, k is a positive integer, such as 1, 2, 3 . . . , there may be one or more k values, and the specific value of k may be adjusted according to an actual situation, which is not limited herein.

When encoding the candidate RNA sequence, the candidate RNA sequence may be converted into N base k-mer subsequences. For example, according to the value of k, consecutive k bases are sequentially taken from the first base of the candidate RNA sequence to form a base k-mer subsequence of the candidate RNA sequence until the last k bases in the candidate RNA sequence are taken, so as to obtain all base k-mer subsequences of the candidate RNA sequence. Each base k-mer subsequence may then be vectorized to obtain N base k-mer vectors, and an RNA vector sequence is constituted by the N base k-mer vectors. For example, the candidate RNA sequence may be divided into N base k-mer subsequences without overlap. For example, if the candidate RNA sequence is AUCUGAAAU, it may be divided into three base k-mer subsequences, AUC, UGA and AAU respectively. It will be understood that the non-overlapping division of the RNA sequence into a plurality of k-mer subsequences is intended to vectorize the bases in the RNA sequence in a k-mer form. Similarly, in other examples, each base included in the candidate RNA sequence may be vectorized to obtain a plurality of base vectors, and the RNA vector sequence may be constituted by the plurality of base vectors. The candidate RNA sequence may also be divided into P base k-mer subsequences with overlap, each base k-mer subsequence is vectorized to obtain P base k-mer vectors, and the RNA vector sequence will be constituted by the P base k-mer vectors, which is not specifically limited by the present disclosure.

In one exemplary embodiment, after converting the candidate RNA sequence into N base k-mer subsequences, each base k-mer subsequence in the candidate RNA sequence may be encoded to obtain first vectors of the N base k-mer subsequences, and the RNA vector sequence will be constituted by the first vectors of the N base k-mer subsequences. For example, when k=3, there may be 64 base 3-mer subsequences, and the first vector of each base k-mer subsequence may be obtained by One-Hot encoding of each base 3-mer subsequence. One-Hot encoding, also known as one-bit active or effective encoding, uses an N-bit status register to encode N statuses, each status having an independent register bit, and only one bit of the register is effective at any time.

For example, for the i-th base 3-mer subsequence, i.e., the base 3-mer subsequence with index as integer i, a 64-dimensional One-Hot vector may be obtained by encoding, and the i-th element in the vector is set to be 1, and other elements are set to be 0, like [0, 1, 0, 0, . . . , 0]. Similarly, each base 3-mer subsequence may correspond to a base 3-mer One-Hot vector. For another example, when k=1, each base is a base 1-mer subsequence, that is, each base in the candidate RNA sequence may be encoded to obtain a representation vector corresponding to each base. For example, if the candidate RNA sequence includes L bases, for the j-th base, i.e., the base with the index of integer j, an L-dimensional One-Hot vector may be obtained by encoding, and the j-th element in the vector is set to 1, and the other elements are set to 0, so as to obtain the One-Hot vector of the j-th base. In other examples, each base in the candidate RNA sequence may also be encoded as a 4-dimensional One-Hot vector according to the base type. For example, the base A may be represented with One-Hot vector [1, 0, 0, 0], U is represented with [0, 0, 0, 1], G is represented with [0, 1, 0, 0], and C is represented with [0, 0, 1, 0]. Correspondingly, One-Hot vectors of all bases in the candidate RNA sequence may be obtained.

For example, the candidate RNA sequence AUCUGAAAU may include three base 3-mer subsequences of AUC, UGA and AAU which corresponds to three base 3-mer One-Hot vectors are V₁^{R}, V₂^{R} and V₃^{R} respectively, and an RNA vector sequence {V₁^{R}, V₂^{R}, V₃^{R}} may be constituted by the three base 3-mer One-Hot vectors. In an exemplary embodiment of the present disclosure, by performing One-Hot encoding on the base k-mer subsequence, each base k-mer subsequence may be changed into a binary feature, thereby remedying the shortcomings of the classifier in processing attribute data, so that the interaction between an RNA sequence and a protein sequence may be predicted more accurately by using the classifier.

In some embodiments of the present disclosure, each base 3-mer subsequence may be represented by a dense vector, that is, all base 3-mer subsequences are sequentially subjected to Embedding (vector mapping) encoding, each base 3-mer subsequence is represented by a low-dimensional vector, so as to obtain a plurality of corresponding base 3-mer Embedding vectors, and an RNA vector sequence is constituted by the plurality of base 3-mer Embedding vectors. For example, each base 3-mer subsequence in an RNA sequence may be mapped into a vector space by using a Word2vec algorithm, and each base 3-mer subsequence may be represented by one vector in the vector space. The base 3-mer subsequence may also be converted into an Embedding vector by using a Doc2vec algorithm, a Glove algorithm, and the like, and each base 3-mer subsequence may also be encoded by using a BERT (i.e., Bidirectional Encoder Representations from Transformers) pre-trained model to obtain a plurality of corresponding base 3-mer Embedding vectors, which is not specifically limited by the present disclosure. In an exemplary embodiment of the present disclosure, the discrete base k-mer subsequence may be converted into one low-dimensional continuous vector by using the Embedding encoding of the base k-mer subsequence, and each base k-mer subsequence may be better represented by using the continuous vector. Also, the Embedding encoding process is learnable, and similar base k-mer subsequences may be closer in a vector space in a continuous training process, so that class distinction is realized while the base k-mer subsequences are encoded, and the interaction between an RNA sequence and a protein sequence may be predicted more accurately subsequently. Furthermore, the efficiency of interaction prediction is improved to some extent.

In one exemplary embodiment, after converting the candidate RNA sequence into N base k-mer subsequences, each base k-mer subsequence may be encoded to obtain first vectors of the N base k-mer subsequences. The first vectors of the N base k-mer subsequences are sequentially input into a pre-trained recurrent neural network, N base k-mer vectors are output, and the RNA vector sequence is constituted by the N base k-mer vectors. The recurrent neural network may include a Long Short-Term Memory (LSTM) network, a bidirectional recurrent neural network, a Gated Recurrent Unit (GRU) network, and the like.

For example, the first vector may be a One-Hot vector. It will be understood that there are relations between bases in the RNA sequence, in this example, all the base 3-mer One-Hot vectors in the candidate RNA sequence may be regarded as a time sequence, and then each base 3-mer One-Hot vector may be operated by using a recurrent neural network. For example, after obtaining all base 3-mer One-Hot vectors (V₁^{R}, V₂^{R} [symbol] V₃^{R}) in the candidate RNA sequence AUCUGAAAU, the three base 3-mer One-Hot vectors may be input into the trained LSTM network, each corresponding base 3-mer vector is output, which is h₁^{R}, h₂^{R} and h₃^{R} respectively, and the RNA vector sequence {h₁^{R}, h₂^{R}, h₃^{R}} is constituted by the three base 3-mer vectors. The LSTM network is a time recurssion neural network suitable for processing and predicting important events with relatively long intervals and delays in the time sequence.

In one exemplary embodiment, after converting the candidate RNA sequence into N base k-mer subsequences, each base k-mer subsequence may be encoded to obtain first vectors of N base k-mer subsequences. Operation on the first vectors of the N base k-mer subsequences is performed by using a first mapping matrix to obtain second vectors of the N base k-mer subsequences, and the RNA vector sequence is constituted by the second vectors of the N base k-mer subsequences.

For example, the first vector may be a One-Hot vector and the second vector may be an Embedding vector. The candidate RNA sequence AUCUGAAAU may include three base 3-mer subsequences, AUC, UGA and AAU, and One-Hot encoding may be performed on the three base 3-mer subsequences to obtain base 3-mer One-Hot vectors which are respectively V₁^{R}, V₂^{R} and V₃^{R}. Since the base 3-mer One-Hot vector is a 64-dimensional sparse vector, the base 3-mer One-Hot vector may be mapped to a dense Embedding vector by the first mapping matrix W₁, that is, the i-th base 3-mer Embedding vector Eᵢ^{R} in the candidate RNA sequence is obtained according to:

$${E}_{i}^{R} = {W}_{1} \times {V}_{i}^{R} \qquad ( 1 )$$

where Vᵢ^{R} represents the i-th base 3-mer One-Hot vector in the candidate RNA sequence, and the first mapping matrix W₁ is a parameter matrix of A*64, for example, A may be 128 or 256, which is not specifically limited by the present disclosure. Based on this, 3-mer Embedding vectors corresponding to the three base 3-mer subsequences may be obtained sequentially, and are respectively E₁^{R}, E₂^{R} and E₃^{R}, and then the RNA vector sequence {E₁^{R}, E₂^{R}, E₃^{R}} will be constituted by the three base 3-mer Embedding vectors.

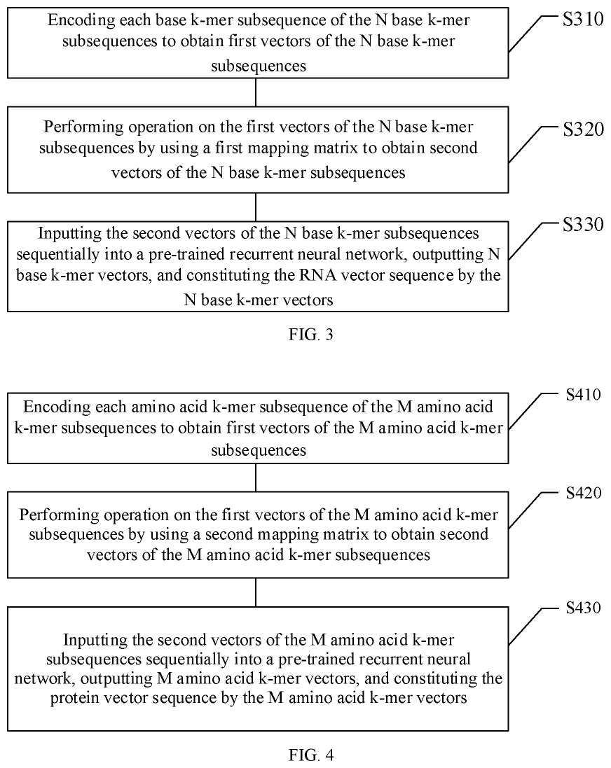

In one exemplary embodiment, after converting the candidate RNA sequence into N base k-mer subsequences, in order to obtain an RNA vector sequence, each base k-mer subsequence may be encoded according to steps S310 to S330, as shown in FIG. 3.

Step S310, encoding each base k-mer subsequence of the N base k-mer subsequences to obtain first vectors of N base k-mer subsequences.

For example, the first vector may be a One-Hot vector. The candidate RNA sequence AUCUGAAAU may include three base 3-mer subsequences, AUC, UGA and AAU, and One-Hot encoding may be performed on the three base 3-mer subsequences to obtain base 3-mer One-Hot vectors which are V₁^{R}, V₂^{R} and V₃^{R}, respectively.

Step S320, performing operation on the first vectors of the N base k-mer subsequences by using a first mapping matrix to obtain second vectors of the N base k-mer subsequences.

The second vector may be an Embedding vector. Since the base 3-mer One-Hot vector is a 64-dimensional sparse vector, the base 3-mer One-Hot vector may be mapped into a dense Embedding vector through a first mapping matrix W₁ to obtain three base 3-mer Embedding vectors which are E₁^{R}, E₂^{R} and E₃^{R}, respectively.

Step S330, inputting the second vectors of the N base k-mer subsequences sequentially into a pre-trained recurrent neural network, outputting N base k-mer vectors, and constituting the RNA vector sequence by the N base k-mer vectors.

It will be understood that there are relations between bases in the RNA sequence, in this example, all base 3-mer Embedding vectors in the candidate RNA sequence may be regarded as one time sequence. In turn, each base 3-mer Embedding vector may be operated by using a recurrent neural network. For example, after obtaining all the base 3-mer Embedding vectors (E₁^{R}, E₂^{R} and E₃^{R}) in the candidate RNA sequence AUCUGAAAU, the three base 3-mer Embedding vectors may be sequentially input into the trained LSTM network, each corresponding base 3-mer vector is output, which is h₁^{R}, h₂^{R} and h₃^{R} respectively, and the RNA vector sequence {h₁^{R}, h₂^{R}, h₃^{R}} will be constituted by the three base 3-mer vectors.

Specifically, the Embedding vector E₁^{R} corresponding to “AUC” may be first input into the LSTM network, implicit feature of E₁^{R} may be extracted through the LSTM network, and the implicit vector h₁^{R} at the time point, such as the time point t, may be output. Then, the implicit vector h₁^{R} at the time point t and the Embedding vector E₂^{R} corresponding to the UGA at the time point t+1 may be spliced, the spliced vector is input into the LSTM network, the implicit feature of the spliced vector is extracted, and the implicit vector h₂^{R} at the time point t+1 is output. Similarly, the Embedding vector at the current time point and the implicit vector transmitted at the previous time point may be spliced in sequence, and feature extraction is performed on the spliced vector through the LSTM network. Finally, the Embedding vector E₃^{R} corresponding to “AAU” may be input into the LSTM network, the implicit vector h₂^{R} at the time point t+1 is spliced with the Embedding vector E₃^{R}, the spliced vector is subjected to implicit feature extraction through the LSTM network, and the implicit vector h₃^{R} at the last time point is output. In other examples, each base 3-mer Embedding vector may be calculated by using a GRU network, and the structure of the GRU network is simpler and the realization effect is the same as the realization effect of the LSTM network. Each base 3-mer One-Hot vector in the candidate RNA sequence may also be directly input into the GRU network to obtain a corresponding base 3-mer vector, which is not specifically limited by the present disclosure.

In this embodiment, when processing a plurality of base 3-mer Embedding vectors in the candidate RNA sequence by using the LSTM network, the dependency relationships between every two base 3-mer Embedding vectors may be learned and memorized to obtain a final RNA vector sequence. When the RNA vector sequence is used for constructing the matching feature matrix, the matching relation between the RNA sequence and the protein sequence may be more accurately embodied, and the accuracy of the RNA-protein interaction prediction may be further improved.

In step S230, the candidate protein sequence is encoded to obtain the protein vector sequence.

After the candidate protein sequence is acquired, the candidate protein sequence may be encoded to obtain a protein vector sequence, and a matching feature matrix will be constructed according to the protein vector sequence and the RNA vector sequence, so that the interaction between the RNA vector sequence and the protein vector sequence will be predicted based on the matching feature matrix.

In one exemplary embodiment, the protein sequence may be represented by an amino acid sequence. There may be 20 amino acids included and the 20 amino acids are sequentially encoded as A, G, V, I, L, F, P, Y, M, T, S, H, N, Q, W, R, K, D, E, C. For example, one protein sequence may be represented as MTAQDDSYS . . . . Correspondingly, the protein sequence may also be represented using amino acid k-mer subsequences. Specifically, all amino acid k-mer subsequences may be obtained by performing permutation and combination on 20 amino acids, and 20^{k} amino acid k-mer subsequences may be obtained for a certain k value. For example, when k is equal to 3, there are a total of 20³=8000 amino acid 3-mer subsequences. For example, MTA, QDD, and SYS are three different amino acid 3-mer subsequences. Thus, the protein sequence MTAQDDSYS . . . may also be denoted as {MTA, QDD, SYS, . . . }. In other examples, the protein sequence may be read in an overlapping manner to obtain the corresponding amino acid 3-mer subsequences. Correspondingly, the amino acid 3-mer subsequences of the protein sequence may further include MTA, TAQ, AQD, and the like. According to the physicochemical properties of the amino acids, the 20 amino acids may also be classified into 7 kinds of {A, G, V}, {I, L, F, P}, {Y, M, T, S}, {H, N, Q, W}, {R, K}, {D, E}, and {C}, and each type of amino acid is recoded, for example, sequentially encoded into 1, 2, 3, 4, 5, 6, and 7. For example, the protein sequence MTAQDDSYS . . . may be converted into 331466333 . . . . Then, all the amino acid k-mer subsequences may be obtained by performing permutation and combination on 7 kinds of amino acids, and 7^{k} kinds of amino acid k-mer subsequences may be obtained for a certain k value, which is not specifically limited by the present disclosure. It will be understood that the classification of 20 amino acids into 7 kinds is merely illustrative, and that 20 amino acids may be classified according to their constituent components. Similarly, the 4 bases of the RNA sequence may be classified according to actual needs.

When encoding the candidate protein sequence, the candidate protein sequence may be converted into M amino acid k-mer subsequences. For example, according to the value of k, consecutive k amino acids are sequentially taken from the first amino acid of the candidate protein sequence to constitute an amino acid k-mer subsequence of the candidate protein sequence until the last k amino acids in the candidate protein sequence are taken, so as to obtain all amino acid k-mer subsequences of the candidate protein sequence. Then, each amino acid k-mer subsequence may be vectorized to obtain M amino acid k-mer vectors, and a protein vector sequence may be constituted by the M amino acid k-mer vectors. For example, the candidate protein sequence may be divided into M amino acid k-mer subsequences without overlap. For example, if the candidate protein sequence is MTAQDDSYS, it may be divided into three amino acid k-mer subsequences, MTA, QDD and SYS. Similarly, in other examples, each amino acid included in the candidate protein sequence may be vectorized to obtain a plurality of amino acid vectors, and the protein vector sequence may be constituted by the plurality of amino acid vectors. The candidate protein sequence may also be divided into Q amino acid k-mer subsequences in an overlapping manner, each amino acid k-mer subsequence is vectorized to obtain Q amino acid k-mer vectors, and the protein vector sequence is constituted by the Q amino acid k-mer vectors, which is not specifically limited by the present disclosure.

In one exemplary embodiment, after converting the candidate protein sequence into M amino acid k-mer subsequences, each amino acid k-mer subsequence in the candidate protein sequence may be encoded to obtain first vectors of the M amino acid k-mer subsequences, and the protein vector sequence is constituted by the first vectors of the M amino acid k-mer subsequences. For example, when k=3, there may be 8000 amino acid 3-mer subsequences, and One-Hot encoding may be performed on each amino acid 3-mer subsequence to obtain the first vector of the amino acid k-mer subsequence.

For example, for the j-th amino acid 3-mer subsequence, i.e., the amino acid 3-mer subsequence with the index of integer j, a 8000-dimensional One-Hot vector may be obtained by encoding, the j-th element in this vector is set to be 1, and other elements are set to be 0, which is in the form of [1, 0, 0, . . . , 0]. Similarly, each amino acid 3-mer subsequence may correspond to one amino acid 3-mer One-Hot vector. For another example, when k=1, each amino acid is an amino acid 1-mer subsequence, that is, each amino acid in the candidate protein sequence may be encoded to obtain a representation vector corresponding to each amino acid. For example, if the candidate protein sequence contains S amino acids, for the j-th amino acid, i.e., the amino acid with the index of integer j, an S-dimensional One-Hot vector may be obtained by encoding, and the j-th element in the vector is set to 1, and the other elements are set to 0, so that the One-Hot vector of the j-th amino acid may be obtained. In other examples, each amino acid in the candidate protein sequence may also be encoded as a 20-dimensional One-Hot vector according to the amino acid type, so as to obtain the One-Hot vectors of all amino acids in the candidate protein sequence. The 20 amino acids may also be classified, and each amino acid in the candidate protein sequence is encoded into the One-Hot vector with the vector dimension consistent with the number of classification categories. For example, when 20 amino acids are classified into 7 kinds, each amino acid in the candidate protein sequence may be encoded as a 7-dimensional One-Hot vector, which is not specifically limited by the present disclosure.

For example, the candidate protein sequence MTAQDDSYS may include three amino acid 3-mer subsequences, MTA, QDD and SYS, which correspond to three amino acid 3-mer One-Hot vectors V₁^{P}, V₂^{P} and V₃^{P} respectively, and the RNA vector sequence {V₁^{P}, V₂^{P}, V₃^{P}} may be constituted by the three amino acid 3-mer One-Hot vectors. In an exemplary embodiment of the present disclosure, by performing One-Hot encoding on the amino acid k-mer subsequences, each k-mer subsequence may be changed into a binary feature, thereby remedying the shortcomings of the classifier in processing attribute data, so that the interaction between a RNA sequence and a protein sequence may be predicted more accurately by using the classifier.

In some embodiments of the present disclosure, each amino acid 3-mer subsequence may be represented by a dense vector, that is, all amino acid 3-mer subsequences are sequentially subjected to Embedding encoding, each amino acid 3-mer subsequence is represented by a low-dimensional vector, so as to obtain a plurality of corresponding amino acid 3-mer Embedding vectors, and a protein vector sequence is constituted by the plurality of amino acid 3-mer Embedding vectors. For example, each amino acid 3-mer subsequence in a protein sequence may be mapped into a vector space by using the Word2vec algorithm, and each amino acid 3-mer subsequence may be represented by one vector in the vector space. The amino acid 3-mer subsequence may also be converted into an Embedding vector by using a Doc2vec algorithm, a Glove algorithm, and the like, and each amino acid 3-mer subsequence may also be encoded by using a BERT pre-trained model to obtain a plurality of corresponding amino acid 3-mer Embedding vectors, which is not specifically limited by the present disclosure. In an exemplary embodiment of the present disclosure, the discrete amino acid k-mer subsequence may be converted to one low-dimensional continuous vector by using the Embedding encoding of the amino acid k-mer subsequence, and each amino acid k-mer subsequence may be better represented by using the continuous vector. Also, the Embedding encoding process is learnable, and similar amino acid k-mer subsequences may be closer in a vector space in a continuous training process, so that the class distinction is realized while the amino acid k-mer subsequences are encoded, and the interaction between an RNA sequence and a protein sequence may be predicted more accurately subsequently. In addition, the efficiency of interaction prediction is improved to some extent.

In one exemplary embodiment, after converting the candidate protein sequence into M amino acid k-mer subsequences, each amino acid k-mer subsequence may be encoded to obtain first vectors of the M amino acid k-mer subsequences. The first vectors of the M amino acid k-mer subsequences are sequentially input into a pre-trained recurrent neural network, M amino acid k-mer vectors are output, and the protein vector sequence is constituted by the M amino acid k-mer vectors.

For example, the first vector may be a One-Hot vector. It will be understood that there are relations between amino acids in the protein sequence, in this example, all the amino acid 3-mer One-Hot vectors in the candidate protein sequence may be regarded as a time sequence, and then each amino acid 3-mer One-Hot vector may be operated by using a recurrent neural network. For example, after obtaining all amino acid 3-mer One-Hot vectors (V₁^{P}, V₂^{P} and V₃^{P}) in the candidate protein sequence MTAQDDSYS, the three amino acid 3-mer One-Hot vectors may be sequentially input into the trained LSTM network, each corresponding amino acid 3-mer vector is output, which is h₁^{P}, h₂^{P} and h₃^{P} respectively, and the protein vector sequence {h₁^{P}, h₂^{P}, h₃^{P}} is constituted by the three amino acid 3-mer vectors.

In one exemplary embodiment, after converting the candidate protein sequence into M amino acid k-mer subsequences, each amino acid k-mer subsequence may be encoded to obtain first vectors of the M amino acid k-mer subsequences. Operation on the first vectors of the M amino acid k-mer subsequences is performed by using a second mapping matrix to obtain second vectors of the M amino acid k-mer subsequences, and a protein vector sequence is constituted by the second vectors of the M amino acid k-mer subsequences.

For example, the first vector may be a One-Hot vector and the second vector may be an Embedding vector. The candidate protein sequence MTAQDDSYS may include three amino acid 3-mer subsequences, MTA, QDD and SYS, and One-Hot encoding may be performed on the three amino acid 3-mer subsequences to obtain amino acid 3-mer One-Hot vectors which are respectively V₁^{P}, V₂^{P} and V₃^{P}. Since the amino acid 3-mer One-Hot vector is a 8000-dimensional sparse vector, the amino acid 3-mer One-Hot vector may be mapped to a dense Embedding vector by the second mapping matrix W₂, that is, the j-th amino acid 3-mer Embedding vector Eᵢ^{P} in the candidate protein sequence is obtained according to:

$${E}_{j}^{P} = {W}_{2} \times {V}_{j}^{P} \qquad ( 2 )$$

where Vⱼ^{P} represents the j-th amino acid 3-mer One-Hot vector in the candidate protein sequence, and the second mapping matrix W₂ is a parameter matrix of B*8000, for example, B may be 256 or 128, which is not specifically limited by the present disclosure. Based on this, 3-mer Embedding vectors corresponding to the three amino acid 3-mer subsequences may be obtained sequentially, and are respectively E₁^{P}, E₂^{P} and E₃^{P}, and then the protein vector sequence {E₁^{P}, E₂^{P}, E₃^{P}} will be constituted by the three amino acid 3-mer Embedding vectors.

In one exemplary embodiment, after converting the candidate protein sequence into M amino acid k-mer subsequences, in order to obtain a protein vector sequence, each amino acid k-mer subsequence may be encoded according to steps S410 to S430, as shown in FIG. 4.

Step S410, encoding each amino acid k-mer subsequence of the M amino acid k-mer subsequences to obtain first vectors of the M amino acid k-mer subsequences.

For example, the first vector may be a One-Hot vector. The candidate protein sequence MTAQDDSYS may include three amino acid 3-mer subsequences, MTA, QDD and SYS, and One-Hot encoding may be performed on the three amino acid 3-mer subsequences to obtain amino acid 3-mer One-Hot vectors which are V₁^{P}, V₂^{P} and V₃^{P}, respectively.

Step S420, performing operation on the first vectors of the M amino acid k-mer subsequences by using a second mapping matrix to obtain second vectors of the M amino acid k-mer subsequences.

The second vector may be an Embedding vector. Since the amino acid 3-mer One-Hot vector is a 8000-dimensional sparse vector, the amino acid 3-mer One-Hot vector may be mapped into a dense Embedding vector through a second mapping matrix W₂ to obtain three amino acid 3-mer Embedding vectors which are E₁^{P}, E₂^{P} and E₃^{P}, respectively.

Step S430, inputting the second vectors of the M amino acid k-mer subsequences sequentially into a pre-trained recurrent neural network, outputting M amino acid k-mer vectors, and constituting the protein vector sequence by the M amino acid k-mer vectors.

In this example, all amino acid 3-mer Embedding vectors in the candidate protein sequence may be regarded as a time sequence, and then each amino acid 3-mer Embedding vector may be operated by using a recurrent neural network. For example, after obtaining all the amino acid 3-mer Embedding vectors (E₁^{P}, E₂^{P} and E₃^{P}) in the candidate protein sequence MTAQDDSYS, the three amino acid 3-mer Embedding vectors may be sequentially input into the trained LSTM network, each corresponding amino acid 3-mer vector is output, which is h₁^{P}, h₂^{P} and h₃^{P} respectively, and the protein vector sequence {h₁^{P}, h₂^{P}, h₃^{P}} will be constituted by the three amino acid 3-mer vectors.

Specifically, the Embedding vector E₁^{P} corresponding to “MTA” may be first input into the LSTM network, implicit feature of E₁^{P} may be extracted through the LSTM network, and the implicit vector h₁^{P} at the time point, such as the time point t, may be output. Then, the implicit vector h₁^{P} at the time point t and the Embedding vector E₂^{P} corresponding to the “QDD” at the time point t+1 may be spliced, the spliced vector is input into the LSTM network, the implicit feature of the spliced vector is extracted, and the implicit vector h₂^{P} at the time point t+1 is output. Finally, the Embedding vector E₃^{P} corresponding to “SYS” may be input into the LSTM network, the implicit vector h at the time point t+1 is spliced with the Embedding vector E₃^{P}, the spliced vector is subjected to implicit feature extraction through the LSTM network, and the implicit vector h₃^{R} at the last time point is output. In other examples, each amino acid 3-mer Embedding vector may be calculated by using a GRU network. Each amino acid 3-mer One-Hot vector in the candidate protein sequence may also be directly input into the GRU network to obtain a corresponding amino acid 3-mer vector, which is not specifically limited by the present disclosure.

In this embodiment, when processing a plurality of amino acid 3-mer Embedding vectors in the candidate protein sequence by using the LSTM network, the dependency relationships among the amino acid 3-mer Embedding vectors may be learned and memorized to obtain a final protein vector sequence. When the protein vector sequence is used for constructing the matching feature matrix, the matching relation between the protein sequence and the RNA sequence may be more accurately embodied, and the accuracy of the RNA-protein interaction prediction may be further improved.

In step S240, the matching feature matrix is constructed according to the RNA vector sequence and the protein vector sequence.

In an exemplary embodiment of the present disclosure, the RNA vector sequence and the protein vector sequence may be used to construct a matching feature matrix, and the matching feature matrix may be used to predict the interaction between the input RNA sequence and the input protein sequence, so that the accuracy of RNA-protein interaction prediction may be improved.

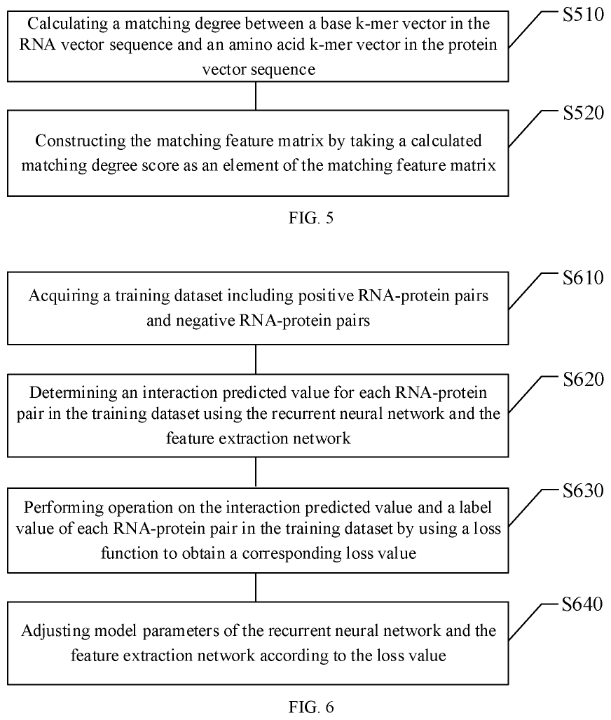

For example, referring to FIG. 5, a matching feature matrix may be constructed by using the RNA vector sequence and the protein vector sequence according to steps S510 and S520.

Step S510, calculating a matching degree between a base k-mer vector in the RNA vector sequence and an amino acid k-mer vector in the protein vector sequence.

When the candidate RNA sequence is converted into N base k-mer subsequences, the corresponding RNA vector sequence may be {hᵢ^{R}, i=1,2, . . . , N}, which includes N base k-mer vectors. Similarly, when the candidate protein sequence is converted into M amino acid k-mer subsequences, the corresponding protein vector sequence may be {hⱼ^{P}, j=1,2, . . . , M}, which includes M amino acid k-mer vectors. It will be understood that the distance between two vectors is negatively correlated to the matching degree between the two vectors. Correspondingly, in an exemplary embodiment of the present disclosure, the larger the distance between the base k-mer vector hᵢ^{R} and the amino acid k-mer vector hⱼ^{P} is, the lower the matching degree between the i-th base k-mer subsequence and the j-th amino acid k-mer subsequence is. The smaller the distance between the base k-mer vector hᵢ^{R} and the amino acid k-mer vector hⱼ^{P} is, the higher the matching degree between the i-th base k-mer subsequence and the j-th amino acid k-mer subsequence is.

In one example, the distance between the i-th base k-mer vector hᵢ^{R} in the RNA vector sequence and the j-th amino acid k-mer vector hⱼ^{P} in the protein vector sequence may be calculated according to:

$$m ( {h}_{i}^{R} , {h}_{j}^{p} ) = exp ( - {‖ \frac{{h}_{i}^{R}}{\vert {h}_{i}^{R} \vert} - \frac{{h}_{j}^{p}}{\vert {h}_{j}^{p} \vert} ‖}_{2} ) \qquad ( 3 )$$

in turn, the matching degree m(hᵢ^{R}, hⱼ^{P}) between hᵢ^{R} and hⱼ^{P} is obtained. Here, |hᵢ^{R}| represents the length of hⱼ^{P}, |hⱼ^{P}| represents the length of hⱼ^{P}, and ∥·∥₂ represents the L2 Norm. Based on this, the matching degrees between N base k-mer vectors and M amino acid k-mer vectors may be calculated sequentially to obtain N*M matching degree scores. The matching degrees between part of the base k-mer vectors and part of the amino acid k-mer vectors may also be calculated, for example, the matching degrees between X base k-mer vectors (N/2<X≤N) and Y amino acid k-mer vectors (M/2<Y≤M) are calculated to obtain X*Y matching degree scores, which is not specifically limited by the present disclosure. It will be understood that, by calculating the matching degrees between most of the base k-mer vectors in the RNA vector sequence and most of the amino acid k-mer vectors in the protein vector sequence, the matching relationship between the RNA vector sequence and the protein vector sequence may be more accurately reflected.

In some embodiments of the present disclosure, the matching degree, between the base k-mer vector in the RNA vector sequence and the amino acid k-mer vector in the protein vector sequence, may also be determined by calculating the Euclidean distance, the Manhattan distance, the Mahalanobis distance, etc. between the base k-mer vector in the RNA vector sequence and the amino acid k-mer vector in the protein vector sequence, so that the matching feature matrix will be constructed, which is not specifically limited by the present disclosure.

Step S520, constructing the matching feature matrix by taking a calculated matching degree score as an element of the matching feature matrix.

For example, when N*M matching degree scores are obtained by calculating the matching degree between a base k-mer vector in the RNA vector sequence and an amino acid k-mer vector in the protein vector sequence, a matching feature matrix F with a size of N*M may be constructed by using each matching degree score as an element of the matching feature matrix. Each element F_{i,j}(i=1,2, . . . , N; j=1,2, . . . , M) in the matching feature matrix F is a matching degree score of the i-th element (base k-mer vector hᵢ^{R}) in the RNA vector sequence and the j-th element (amino acid k-mer vector hⱼ^{P}) in the protein vector sequence, and it may represent the matching degree between the i-th base k-mer vector hᵢ^{R} in the RNA vector sequence and the j-th amino acid k-mer vector hⱼ^{P} in the protein vector sequence.

In some embodiments of the present disclosure, the matching feature matrix may also be obtained by performing a dot-product operation on the RNA vector sequence and the protein vector sequence. For example, a dot-product operation may be performed on the RNA vector sequence {hᵢ^{R}, i=1,2, . . . , N} and the protein vector sequence {hⱼ^{P}, j=1,2, . . . , M}. For example, the RNA vector sequence may be first transposed, and a dot-product operation is performed on the transposed RNA vector sequence and the protein vector sequence, to obtain a matching feature matrix including N*M elements, where each element may correspond to a matching degree score between a base k-mer vector and an amino acid k-mer vector. The protein vector sequence may also be first transposed, and then a dot-product operation is performed on the transposed protein vector sequence and the RNA vector sequence. In some cases, the transpose operation may also be omitted if one of the protein vector sequence and the RNA vector sequence is a row vector and the other is a column vector.

In an exemplary embodiment of the present disclosure, a matching feature matrix is constructed by using an RNA vector sequence and a protein vector sequence, and the matching feature matrix can accurately represent a matching relationship between the RNA vector sequence and the protein vector sequence, so that when an interaction between the input RNA sequence and the input protein sequence is predicted by using the matching feature matrix, the accuracy of RNA-protein interaction prediction may be improved.

In step S250, the feature extraction is performed on the matching feature matrix, and the interaction between the RNA sequence and the candidate protein sequence is determined according to the extracted matching feature.

In an exemplary embodiment of the present disclosure, the interaction between the candidate RNA sequence and the candidate protein sequence needs to be predicted, and the obtained prediction result may be the presence of an interaction between the candidate RNA sequence and the candidate protein sequence, or may be the absence of an interaction between the candidate RNA sequence and the candidate protein sequence, that is, a binary classification prediction. Feature extraction may be performed on the matching feature matrix by using a feature extraction network, and whether the interaction exists between the candidate RNA sequence and the candidate protein sequence may be determined according to the extracted matching feature. The feature extraction network may be a convolutional neural network. For example, a convolutional neural network may be used to perform feature extraction on the matching feature matrix F to obtain the matching feature.

In an exemplary embodiment of the present disclosure, the network structure of the convolutional neural network may include at least one convolutional layer and at least one pooling layer. After the matching feature matrix F is input into the convolutional neural network, the feature matrix FM with a size of D*E may be output. In some embodiments, for convenience of performing the following binary classification prediction, the feature matrix FM may be subjected to a dimensionality reduction processing, for example, the feature matrix FM may be flattened and converted into a D×E-dimensional feature vector, and the feature vector is subjected to classification prediction by using a classifier, so as to obtain a predicted value of an interaction between a candidate RNA sequence and a candidate protein sequence.

Since the lengths of different RNA sequences may be different, i.e., the number of bases contained may be different, and the lengths of different protein sequences may also be different, the length of the sequence used for prediction may be set to 1000 in order to improve the processing efficiency, i.e., N=M=1000. Correspondingly, the size of the matching feature matrix F is 1000×1000. If the sequence length is less than 1000, zero padding may be performed. If the sequence length exceeds 1000, the first 1000 elements may be selected from the sequence. In an exemplary embodiment of the present disclosure, the network structure of the convolutional neural network may be: 6 convolutional layers of 5×5, i.e., with a convolutional kernel size of 5×5 per convolutional layer, an average pooling layer of 3×3, a Rectified Linear Unit (ReLU) active layer; 6 convolutional layers of 5×5, an average pooling layer of 4×4, a ReLU active layer; 6 convolutional layers with a convolution kernel size of 3×3 per convolutional layer, an average pooling layer of 4×4, a ReLU active layer. After inputting a matching feature matrix F of size 1000*1000 into the convolutional neural network, a feature matrix FM of size 20*20 may be output.

Further, the 20*20 feature matrix FM may be flattened into a 400-dimensional feature vector ν. For example, all elements FM_{0,0}, FM_{0,1}, . . . FM_{19,19} in the feature matrix FM may be arranged sequentially in a row or a column, and when arranged in a row, a 400-dimensional feature vector is obtained as:

$$v = [ {FM}_{0 , 0} , … , {FM}_{0 , 19} , {FM}_{1 , 0} , … , {FM}_{1 , 19} , … , {FM}_{19 , 19} ] .$$

In some embodiments, the convolutional neural network may be used to perform feature extraction on the matching feature matrix F to obtain an original feature, and a third mapping matrix is used to perform operation on the original feature to obtain the matching feature.

In order to perform the binary classification prediction, the original feature extracted by the convolutional neural network need to be subjected to dimensionality reduction processing, for example, the original feature may be subjected to dimensionality reduction processing through a fully connected layer to obtain the matching feature for performing interaction prediction. Correspondingly, a matching feature c may be obtained by using the third mapping matrix to operate on the feature vector, i.e. according to:

$$c = {W}_{3} \times v ; \qquad ( 4 )$$

where c is a 2-dimensional feature vector [c₀, c₁], the third mapping matrix W₃ is a parameter matrix of 2*C, ν is a feature vector, and the value of C is consistent with the dimension of the feature vector.

In other examples, after the 1000×1000 matching feature matrix F is input into the convolutional neural network, the feature vector may also be directly output by using a specific convolutional neural network, that is, the matching feature used for performing the interaction prediction is obtained, for example, a 2-dimensional feature vector, in this case, the above dimensionality reduction step may be omitted, which is not specifically limited by the present disclosure. In an exemplary embodiment of the present disclosure, the convolutional neural network is used to perform feature extraction on the matching feature matrix, and when the extracted invariant matching features are used to perform interaction prediction, the accuracy of RNA-protein interaction prediction may be improved.

In one exemplary embodiment, after the matching feature for interaction prediction is obtained, an interaction predicted value between the candidate RNA sequence and the candidate protein sequence may be obtained according to the matching feature, and the interaction between the candidate RNA sequence and the candidate protein sequence may be determined according to the interaction predicted value. For example, the matching feature may be input into a classifier, and the interaction between the candidate RNA sequence and the candidate protein sequence may be classified according to the matching feature. After the classification is completed, the predicted value of the interaction between the candidate RNA sequence and the candidate protein sequence is output. For example, the interaction between the candidate RNA sequence and the candidate protein sequence may be predicted by using a Softmax classifier. Specifically, the matching feature may be transformed by using the Softmax classifier to obtain a probability distribution of “presence of an interaction” and a probability distribution of “absence of interaction” to which interactions between the candidate RNA sequence and the candidate protein sequence belong respectively.

For example, the probability of the presence of an interaction between the candidate RNA sequence and the candidate protein sequence may be obtained by a Softmax classifier is:

$$P ( 1 \vert r , p ) = \frac{{e}^{{c}_{1}}}{{e}^{{c}_{1}} + {e}^{{c}_{0}}} . \qquad ( 5 )$$

And the probability of the absence of an interaction between the RNA sequence and the candidate protein sequence is:

$$P ( 0 \vert r , p ) = \frac{{e}^{{c}_{0}}}{{e}^{{c}_{1}} + {e}^{{c}_{0}}} . \qquad ( 6 )$$

Here, r represents the candidate RNA sequence, p represents the candidate protein sequence, c₀ is a first feature value of the matching feature, and c₁ represents a second feature value of the matching feature. When the matching feature is a 2-dimensional vector, the vector is (c₀, c₁). In other examples, the binary classification prediction may also be performed by using a logistic regression classifier or a Support Vector Machine (SVM) classifier, so as to obtain the predicted value of the interaction between the candidate RNA sequence and the candidate protein sequence according to the matching feature, which is not specifically limited by the present disclosure.

After a classifier is used to obtain the predicted value of the interaction between the candidate RNA sequence and the candidate protein sequence, the interaction between the candidate RNA sequence and the candidate protein sequence may be determined according to the predicted value of the interaction. For example, if the predicted value of the interaction meets a preset threshold condition, it may be determined that there is an interaction between the candidate RNA sequence and the candidate protein sequence.

For example, the probability P(1|r, p) of the presence of an interaction between the candidate RNA sequence and the candidate protein sequence may be obtained using the Softmax classifier. P(1|r, p) may be any value between 0 and 1. For example, a threshold value of 0.5 may be preset for the probability of the presence of an interaction, and when P(1|r, p)>0.5, the prediction result may be labeled as 1, i.e., it can be determined that there is an interaction between the candidate RNA sequence and the candidate protein sequence. When P(1|r, p)≤0.5, the prediction result may be labeled as 0, i.e., it can be determined that there is no interaction between the candidate RNA sequence and the candidate protein sequence. In other examples, it may be set that when P(1|r, p)≥0.5, it may be determined that there is an interaction between the candidate RNA sequence and the candidate protein sequence, and when P(1|r, p)<0.5, it may be determined that there is no interaction between the candidate RNA sequence and the candidate protein sequence. Finally, the prediction result of the interaction between the candidate RNA sequence and the candidate protein sequence may be output to the terminal device for the user to view. It should be noted that, only the probability value of the presence of an interaction between the candidate RNA sequence and the candidate protein sequence may be output, only the probability value of the absence of an interaction between the candidate RNA sequence and the candidate protein sequence may be output, or the probability value of the presence of an interaction and the probability value of the absence of an interaction between the candidate RNA sequence and the candidate protein sequence may be output at the same time, which is not specifically limited by the present disclosure.

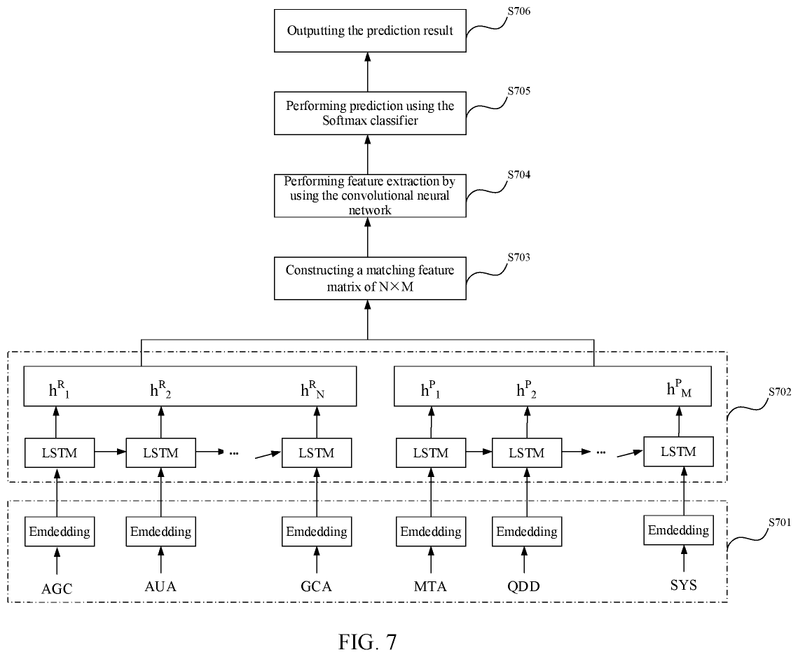

In an exemplary embodiment of the present disclosure, as shown in FIG. 6, the recurrent neural network and the feature extraction network may be pre-trained according to steps S610 to S650, and parameters in each parameter matrix are trained at the same time, so as to optimize all parameters in each prediction model and parameter matrix, and further predict an RNA sequence and a protein sequence with unknown interactions according to a final model obtained by training.

Step S610, acquiring a training dataset including positive RNA-protein pairs and negative RNA-protein pairs.

For example, training of the various models may be performed based on the RPI1807 dataset. There are a total of 3243 RNA-protein pairs in the dataset, specifically, 1807 positive and 1436 negative examples. A positive example may indicate that there is an interaction between the RNA sequence and the protein sequence in the RNA-protein pair, and a negative example may indicate that there is no interaction between the RNA sequence and the protein sequence in the RNA-protein pair. 1200 positive examples and 1000 negative examples may be selected as the training dataset. All the RNA-protein pairs may also be selected as the training dataset. It will be understood that the number of the RNA-protein pairs in the training dataset is merely illustrative and that any number of the RNA-protein pairs may be acquired for multiple training of each model to improve the performance of each model. It should be noted that the positive RNA-protein pair may be labeled, and the obtained label value of “1” indicates that the RNA-protein pair has an interaction. The negative RNA-protein pair may be labeled, and the obtained label value of “0” indicates that the RNA-protein pair has no interaction. It will be understood that experiments may be performed based on a RPI2241 dataset, a RPI369 dataset, and the like in other examples, which are not specifically limited by the present disclosure.

Step S620, determining an interaction predicted value for each RNA-protein pair in the training dataset using the recurrent neural network and the feature extraction network.

Similarly, the RNA sequence and the protein sequence in each RNA-protein pair in the training dataset may be encoded using a recurrent neural network, to obtain the corresponding RNA vector sequence and protein vector sequence. A matching feature matrix corresponding to each RNA-protein pair is constructed according to the RNA vector sequence and the protein vector sequence, and the matching feature matrix is input into a feature extraction network for feature extraction. Finally, classification prediction is performed on the extracted matching feature by using a classifier to obtain the predicted value of the interaction of each RNA-protein pair.

Step S630, performing operation on the interaction predicted value and a label value of each RNA-protein pair in the training dataset by using a loss function to obtain a corresponding loss value.

There is a label value for each RNA-protein pair in the training dataset, e.g., 1 for each positive pair and 0 for each negative pair. For example, the i-th RNA-protein pair is positive data, corresponding to a label value of 1. A loss function may be calculated according to the interaction predicted values of p(1|rᵢ, pᵢ), and p(0|rᵢ, pᵢ), and the label value of 1 of the RNA-protein pair to obtain a corresponding loss value. During the training of the model, it is necessary to make the interaction predicted value infinitely close to the label value, i.e. minimize the target function. In one example, when the target function needs to be minimized, the cross-entropy loss function may be chosen as the target function. When calculating the cross-entropy loss function, if the label value is 1, the closer p(1|rᵢ, pᵢ) is to 1, the smaller the calculated loss value is, and the closer p(1|rᵢ, pᵢ) is to 0, the larger the calculated loss value is. Meanwhile, the closer p(0|rᵢ, pᵢ) is to 1, the larger the calculated loss value is, and the closer p(0|rᵢ, pᵢ) is to 0, the smaller the calculated loss value is. It will be understood that the cross-entropy loss function is a performance function in the prediction model, and may be used to measure the degree of inconsistency between the predicted value and the label value of the prediction model. The smaller the value of the cross-entropy loss function obtained by calculation is, the better the effect of model prediction is represented.

Specifically, the cross-entropy loss function may be:

$$loss = - {\sum }_{i = 1}^{K} ( {y}_{i} log p {( 1 \vert {r}_{i} , {p}_{i} )}^{{y}_{i}} + ( 1 - {y}_{i} ) log {p ( 0 \vert {r}_{i} , {p}_{i} )}^{( 1 - {y}_{i}} ) ; \qquad ( 7 )$$

where rᵢ represents the i-th RNA sequence in the training dataset, pᵢ represents the i-th protein sequence in the training dataset, yᵢ represents the label value of the i-th RNA-protein pair in the training dataset, p(1|rᵢ,pᵢ) represents the predicted value of the presence of an interaction for the i-th RNA-protein pair in the training dataset, p(0|rᵢ, pᵢ) represents the predicted value of the absence of an interaction of the i-th RNA-protein pair in the training dataset, and K is the total number of the RNA-protein pairs in the training dataset.

Step S640, adjusting model parameters of the recurrent neural network and the feature extraction network according to the loss value.

The model parameters may be weight parameters, offset parameters, parameter matrices, i.e., the mapping matrices W₁, W₂ and W₃, and the like. For example, model parameter of each model may be iteratively updated based on the calculated loss value, and when an iteration termination condition is met, model parameter training of a plurality of interaction prediction models is completed. For example, the model parameter may be updated using a stochastic gradient descent algorithm. According to the back propagation principle, a target function such as a cross-entropy loss function is continuously calculated, and the model parameter of each model is updated simultaneously according to the calculated loss value. When the target function is converged to the minimum value, the training of all model parameters is completed. The model parameter may also be updated in a reverse iterative manner, and when the preset iteration times are met, the training of all the model parameters is completed. After the iteration is completed, optimized model parameter may be obtained. In other examples, the target function may be minimized by an alternating least square method, Adam optimization algorithm, or the like, and the model parameters may be updated sequentially from back to front to optimize the parameters.

In the above training process, parameters in the recurrent neural network and the feature extraction network may be trained simultaneously. For example, taking L as the target function, the mapping matrix W₃ in the fully connected layer may be adjusted first. Because the convolutional neural network is required to be used for performing feature extraction on the matching feature matrix before the binary classification prediction is performed, and the recurrent neural network is required to be used for encoding the candidate RNA sequence and the candidate protein sequence, further back propagation may be performed the convolutional neural network and the recurrent neural network, and model parameters and mapping matrices W₁, W₂ in the convolutional neural network and the recurrent neural network are adjusted. Through multiple back propagation layer by layer, each model parameter may be finally tend to converge, or training is terminated after a certain iteration number is met. Through such training mode, the recurrent neural network and the feature extraction network may be trained simultaneously, in order to ensure higher precision and accuracy of each model, and improve training efficiency. After the training is completed, the interaction between the candidate RNA sequence and the candidate protein sequence may be predicted by using each finally obtained model.

In a specific exemplary embodiment, referring to FIG. 7, the interaction between the candidate RNA sequence and the candidate protein sequence may be predicted using a trained LSTM network, a convolutional neural network, and a Softmax classifier according to steps S701 to S706.

Step S701, converting a candidate RNA sequence AGCAUA . . . GCA into N base 3-mer subsequences AGC, AUA, and so on, and performing Embedding encoding on each base 3-mer subsequence to obtain N base 3-mer Embedding vectors; and converting a candidate protein sequence MTAQDD . . . SYS into M amino acid 3-mer subsequences MTA, QDD, and so on, and performing Embedding encoding on each amino acid 3-mer subsequence to obtain M amino acid 3-mer Embedding vectors.

Step S702, inputting the obtained N base 3-mer Embedding vectors and M amino acid 3-mer Embedding vectors into the LSTM network, and outputting the vector hᵢ^{R} corresponding to each base 3-mer Embedding vector and the vector hᵢ^{P} corresponding to each amino acid 3-mer Embedding vector. And the vectors hᵢ^{R} corresponding to the N base 3-mer Embedding vectors form an RNA vector sequence {hᵢ^{R}, h₂^{R}, . . . , h_{N}^{R}}, and the vectors hᵢ^{P} corresponding to the M amino acid 3-mer Embedding vectors form a protein vector sequence {h₁^{P}, h₂^{P}, . . . , h_{M}^{P}}.

Step S703, constructing a matching feature matrix of N×M. The matching feature matrix with the size of N*M is constructed according to the RNA vector sequence {h₁^{R}, h₂^{R}, . . . , h_{N}^{R}} and the protein vector sequence {h₁^{P}, h₂^{P}, . . . , h_{M}^{P}}.

Step S704, performing feature extraction by using the convolutional neural network. The matching feature matrix is input into the convolutional neural network for feature extraction to obtain a matching feature vector.

Step S705, performing prediction by using the Softmax classifier. The binary classification prediction is performed on the matching feature vector by using the Softmax classifier to obtain a predicted value of the interaction between the candidate RNA sequence and the candidate protein sequence.

Step S706, outputting the prediction result. The predicted value of the interaction between the candidate RNA sequence and the candidate protein sequence is output to the terminal device for the user to view.

In this exemplary embodiment, a matching feature matrix is constructed by using the matching relationship between the RNA sequence and the protein sequence, and the matching feature matrix may accurately represent the matching relationship between the RNA vector sequence and the protein vector sequence. Compared with the prior art in which the association among various k-mer subsequences is not considered in the ncRPI prediction process, and each k-mer subsequence is taken as an independent factor, the present disclosure can improve the accuracy of the RNA-protein interaction prediction when predicting the interaction between the RNA sequence and the protein sequence according to the matching feature matrix. It should be noted that the interaction prediction methods provided by the present disclosure are applicable to, but not limited to, predicting the interaction between the RNA sequence and the protein sequence. For example, the interaction between a first substance and a second substance may be predicted by using the interaction prediction method, where the first substance and the second substance may be represented by sequence segments, which is not limited by the present disclosure.

In an exemplary embodiment of the present disclosure, at least one RNA sequence may also be acquired, and protein sequence(s) that interacts with each of the input RNA sequences may be searched in the database. For example, when the user inputs at least one RNA sequence, each input RNA sequence may be combined with all protein sequences in the database into several RNA-protein pairs. Further, the interaction of each RNA-protein pair may be predicted according to steps S220 to S260. Specifically, the RNA sequence and the protein sequence in each RNA-protein pair may be encoded using a recurrent neural network to obtain the corresponding RNA vector sequence and protein vector sequence. A matching feature matrix corresponding to each RNA-protein pair is constructed according to the RNA vector sequence and the protein vector sequence, and the matching feature matrix is input into the feature extraction network for feature extraction. Finally, classification prediction is performed on the extracted matching feature by using a classifier to obtain the predicted value of the interaction of each RNA-protein pair. The predicted value of the interaction of 1 indicates that the RNA-protein pair has an interaction, and the predicted value of the interaction of 0 indicates that the RNA-protein pair has no interaction. Then, all the RNA-protein pairs with the predicted value of the interaction of 1 may be screened out, and the protein sequence in each RNA-protein pair is output to the terminal device for the user to view the protein sequence(s) interacting with the input RNA sequence.

Similarly, in an exemplary embodiment of the present disclosure, at least one protein sequence may also be acquired, and RNA sequence(s) that interacts with each of the input protein sequences may be searched in the database. For example, when the user inputs at least one protein sequence, each input protein sequence may be combined with all RNA sequences in the database into several RNA-protein pairs. Further, the interaction of each RNA-protein pair may be predicted according to steps S220 to S260. Specifically, the RNA sequence and the protein sequence in each RNA-protein pair may be encoded using the recurrent neural network to obtain the corresponding RNA vector sequence and protein vector sequence. A matching feature matrix corresponding to each RNA-protein pair is constructed according to the RNA vector sequence and the protein vector sequence, and the matching feature matrix is input into the feature extraction network for feature extraction. Finally, classification prediction is performed on the extracted matching feature by using a classifier to obtain the predicted value of the interaction of each RNA-protein pair. The predicted value of the interaction of 1 indicates that the RNA-protein pair has an interaction, and the predicted value of the interaction of 0 indicates that the RNA-protein pair has no interaction. Then, all the RNA-protein pairs with the predicted value of the interaction of 1 may be screened out, and the RNA sequence in each RNA-protein pair is output to the terminal device for the user to view the RNA sequence(s) interacting with the input protein sequence.

In the method for predicting the RNA-protein interaction provided by the exemplary embodiments of the present disclosure, a candidate RNA sequence and a candidate protein sequence are acquired; the candidate RNA sequence is encoded to obtain an RNA vector sequence; the candidate protein sequence is encoded to obtain a protein vector sequence; a matching feature matrix is constructed according to the RNA vector sequence and the protein vector sequence; feature extraction is performed on the matching feature matrix, and the interaction between the candidate RNA sequence and the candidate protein sequence is determined according to the extracted matching feature. The present disclosure can improve the accuracy of RNA-protein interaction prediction by: constructing a matching feature matrix by using the matching relationship between the RNA sequence and the protein sequence, and predicting the interaction between the RNA sequence and the protein sequence according to the matching feature matrix.

It should be noted that, although the various steps of the method of the present disclosure are described in a particular order in the accompanying figures, this is not required or implied that the steps must be performed in the particular order, or all the steps shown must be performed to achieve the desired result. Additionally or alternatively, certain steps may be omitted, a plurality of steps may be combined into one step, and/or one step may be decomposed into a plurality of steps and the like.

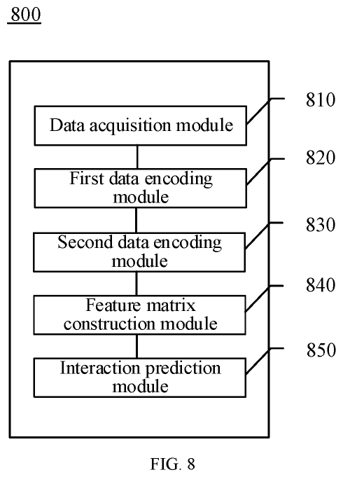

Further, in this exemplary embodiment, an apparatus for predicting an RNA-protein interaction is also provided. The apparatus may be applied to a server or a terminal device. Referring to FIG. 8, the apparatus for predicting the RNA-protein interaction 800 may include a data acquisition module 810, a first data encoding module 820, a second data encoding module 830, a feature matrix construction module 840, and an interaction prediction module 850.

The data acquisition module 810 is configured to acquire a candidate RNA-protein pair including a candidate RNA sequence and a candidate protein sequence.

The first data encoding module 820 is configured to encode the candidate RNA sequence to obtain an RNA vector sequence.

The second data encoding module 830 is configured to encode the candidate protein sequence to obtain a protein vector sequence.

The feature matrix construction module 840 is configured to construct a matching feature matrix according to the RNA vector sequence and the protein vector sequence.

The interaction prediction module 850 is configured to perform feature extraction on the matching feature matrix, and determine, according to an extracted matching feature, an interaction between the candidate RNA sequence and the candidate protein sequence.

In an alternative embodiment, the first data encoding module 820 includes:
a first sequence conversion module configured to convert the candidate RNA sequence into N base k-mer subsequences; and
a first sequence encoding module configured to vectorize each base k-mer subsequence of the N base k-mer subsequences to obtain the RNA vector sequence.

In an alternative embodiment, the first sequence encoding module includes:
a first sequence encoding unit configured to encode each base k-mer subsequence of the N base k-mer subsequences to obtain first vectors of the N base k-mer subsequences; and
a first vector sequence determination unit configured to constitute the RNA vector sequence by the first vectors of the N base k-mer subsequences.

In an alternative embodiment, the first sequence encoding module includes:
a second sequence encoding unit configured to encode each base k-mer subsequence of the N base k-mer subsequences to obtain first vectors of the N base k-mer subsequences; and
a second vector sequence determination unit configured to input the first vectors of the N base k-mer subsequences sequentially into a pre-trained recurrent neural network, output N base k-mer vectors, and constitute the RNA vector sequence by the N base k-mer vectors.

In an alternative embodiment, the first sequence encoding module includes:
a third sequence encoding unit configured to encode each base k-mer subsequence of the N base k-mer subsequences to obtain first vectors of the N base k-mer subsequences; and
a third vector sequence determination unit configured to perform operation on the first vectors of the N base k-mer subsequences by using a first mapping matrix to obtain second vectors of the N base k-mer subsequences, and constituting the RNA vector sequence by the second vectors of the N base k-mer subsequences.

In an alternative embodiment, the first sequence encoding module includes:
a fourth sequence encoding unit configured to encode each base k-mer subsequence of the N base k-mer subsequences to obtain first vectors of the N base k-mer subsequences;
a first vector operation unit configured to perform operation on the first vectors of the N base k-mer subsequences by using a first mapping matrix to obtain second vectors of the N base k-mer subsequences; and
a fourth vector sequence determination unit configured to input the second vectors of the N base k-mer subsequences sequentially into a pre-trained recurrent neural network, output N base k-mer vectors, and constitute the RNA vector sequence by the N base k-mer vectors.

In an alternative embodiment, the second data encoding module 830 include:
the second sequence conversion module configured to convert the candidate protein sequence into M amino acid k-mer subsequences; and
the second sequence encoding module configured to vectorize each amino acid k-mer subsequence of the M amino acid k-mer subsequences to obtain the protein vector sequence.

In an alternative embodiment, the second sequence encoding module includes:
a fifth sequence encoding unit configured to encode each amino acid k-mer subsequence of the M amino acid k-mer subsequences to obtain first vectors of the M amino acid k-mer subsequences; and
a fifth vector sequence determination unit configured to constitute the protein vector sequence by the first vectors of the M amino acid k-mer subsequences.

In an alternative embodiment, the second sequence encoding module includes:
a sixth sequence encoding unit configured to encode each amino acid k-mer subsequence of the M amino acid k-mer subsequences to obtain first vectors of the M amino acid k-mer subsequences; and
a sixth vector sequence determination unit configured to input the first vectors of the M amino acid k-mer subsequences sequentially into a pre-trained recurrent neural network, output M amino acid k-mer vectors, and constitute the protein vector sequence by the M amino acid k-mer vectors.

In an alternative embodiment, the second sequence encoding module includes:
a seventh sequence encoding unit configured to encode each amino acid k-mer subsequence of the M amino acid k-mer subsequences to obtain first vectors of the M amino acid k-mer subsequences; and
a seventh vector sequence determination unit configured to perform operation on the first vectors of the M amino acid k-mer subsequences by using a second mapping matrix to obtain second vectors of the M amino acid k-mer subsequences, and constitute the protein vector sequence by the second vectors of the M amino acid k-mer subsequences.

In an alternative embodiment, the second sequence encoding module includes:
an eighth sequence encoding unit configured to encode each amino acid k-mer subsequence of the M amino acid k-mer subsequences to obtain first vectors of the M amino acid k-mer subsequences;
a second vector operation unit configured to perform operation on the first vectors of the M amino acid k-mer subsequences by using a second mapping matrix to obtain second vectors of the M amino acid k-mer subsequences; and
an eighth vector sequence determination unit configured to input the second vectors of the M amino acid k-mer subsequences sequentially into a pre-trained recurrent neural network, output M amino acid k-mer vectors, and constitute the protein vector sequence by the M amino acid k-mer vectors.

In an alternative embodiment, the feature matrix construction module 840 includes:
a matching degree calculating unit configured to obtain a calculated matching degree score by calculating a matching degree between a base k-mer vector in the RNA vector sequence and an amino acid k-mer vector in the protein vector sequence; and
a feature matrix construction unit configured to construct the matching feature matrix by taking the calculated matching degree score as an element of the matching feature matrix.

In an alternative embodiment, the matching degree calculating unit includes:
a first matching degree calculating subunit configured to calculate the matching degree m(hᵢ^{R}, hⱼ^{P}) between the i-th base k-mer vector hᵢ^{R} in the RNA vector sequence and the j-th amino acid k-mer vector hⱼ^{P} in the protein vector sequence according to:

$$m ( {h}_{i}^{R} , {h}_{j}^{p} ) = exp ( - {‖ \frac{{h}_{i}^{R}}{\vert {h}_{i}^{R} \vert} - \frac{{h}_{j}^{p}}{\vert {h}_{j}^{p} \vert} ‖}_{2} ) ;$$

where |hᵢ^{R}| represents a length of hᵢ^{R}, and |hⱼ^{P}| represents a length of hⱼ^{P}.

In an alternative embodiment, the feature matrix construction module 840 is configured to perform a dot-product operation on the RNA vector sequence and the protein vector sequence to obtain the matching feature matrix.

In an alternative embodiment, an interaction prediction module 850 includes:
a feature extraction module configured to perform feature extraction on the matching feature matrix by using a feature extraction network to obtain the extracted matching feature.

In an alternative embodiment, the feature extraction module includes:
a feature extraction unit configured to perform feature extraction on the matching feature matrix by using the feature extraction network to obtain an original feature; and
a third vector operation unit configured to perform operation on the original feature by using a third mapping matrix to obtain the extracted matching feature.

In an alternative embodiment, the interaction prediction module 850 further includes:
a predicted value acquiring module configured to obtain an interaction predicted value between the candidate RNA sequence and the candidate protein sequence according to the extracted matching feature; and
an interaction determination module configured to determine the interaction between the candidate RNA sequence and the candidate protein sequence according to the interaction predicted value.

In an alternative embodiment, the interaction prediction module 850 further includes:
an interaction prediction module configured to input the extracted matching feature into a classifier, and outputting a probability of the presence of the interaction between the candidate RNA sequence and the candidate protein sequence.

In an alternative embodiment, the probability of the presence of the interaction between the candidate RNA sequence and the candidate protein sequence is

$$P ( 1 \vert r , p ) = \frac{{e}^{{c}_{1}}}{{e}^{{c}_{1}} + {e}^{{c}_{0}}} ;$$

where r represents the candidate RNA sequence, p represents the candidate protein sequence, c₀ represents a first feature value in the extracted matching feature, and c₁ represents a second feature value in the extracted matching feature.

In an alternative embodiment, the interaction determination module is configured to determine, in response to the interaction predicted value meeting a preset threshold condition, that the interaction exists between the candidate RNA sequence and the candidate protein sequence.

In an alternative embodiment, the apparatus for predicting the RNA-protein interaction 800 further includes:
a training module configured to train a recurrent neural network and a feature extraction network.

In an alternative embodiment, the training module includes:
a trained data acquisition module configured to acquire a training dataset including positive RNA-protein pairs and negative RNA-protein pairs;
a predicted value output module configured to determine an interaction predicted value for each RNA-protein pair in the training dataset using the recurrent neural network and the feature extraction network;
a loss value calculating module configured to perform operation on the interaction predicted value and a label value of each RNA-protein pair in the training dataset by using a loss function to obtain a corresponding loss value; and
a model parameter adjustment module configured to adjust model parameters of the recurrent neural network and the feature extraction network according to the loss value.

In an alternative embodiment, the loss function is:

$$loss = - {\sum }_{i = 1}^{K} ( {y}_{i} log {p ( 1 \vert {r}_{i} , {p}_{i} )}^{{y}_{i}} + ( 1 - {y}_{i} ) log {p ( 0 \vert {r}_{i} , {p}_{i} )}^{( 1 - {y}_{i} )}$$

where rᵢ represents the i-th RNA sequence in the training dataset, pᵢ represents the i-th protein sequence in the training dataset, yᵢ represents the label value of the i-th RNA-protein pair in the training dataset, p(1|rᵢ, pᵢ) represents the predicted value of the presence of the interaction for the i-th RNA-protein pair in the training dataset, p(0|rᵢ, pᵢ) represents the predicted value of the absence of the interaction for the i-th RNA-protein pair in the training dataset, and K is the total number of the RNA-protein pairs in the training dataset.

In an alternative embodiment, the model parameter adjustment module is configured to perform iterative update on the model parameters of the recurrent neural network and the feature extraction network by using a stochastic gradient descent algorithm based on the loss value, and complete, in response to meeting an iteration termination condition, the training of the model parameters.

In an alternative embodiment, the apparatus for predicting the RNA-protein interaction 800 further includes:
a data output module configured to output a prediction result of the interaction between the candidate RNA sequence and the candidate protein sequence.

The specific details of each module in the above apparatus for predicting the RNA-protein interaction are described in detail in the corresponding method for predicting the RNA-protein interaction, and thus are not described in detail herein.

Each module in the above apparatus may be a general-purpose processor, including: a central processor, a network processor, etc.; but may also be a digital signal processor, an application-specific integrated circuit, a field programmable gate array or other programmable logic device, discrete gate or transistor logic device, discrete hardware components. Each module may also be implemented in software, firmware, etc. The processors in the above apparatus may be independent processors or may be integrated together.

Exemplary embodiments of the present disclosure also provide a computer-readable storage medium having a program product capable of implementing the above method of the specification stored thereon. In some possible embodiments, various aspects of the present disclosure may also be implemented as a form of a program product, which includes a program code. When the program product runs on the electronic device, the program code is used for enabling the electronic device to perform the steps described in the above sections of the specification according to various exemplary embodiments of the present disclosure. The program product may employ a Compact Disk Read-Only Memory (CD-ROM) and include a program code, and may be run on an electronic device, for example, a personal computer. However, the program product of the present disclosure is not limited thereto, and in this document, a readable storage medium may be any tangible medium that may contain, or store a program for use by or in connection with an instruction execution system, an apparatus, or a device.

The program product may use any combination of one or more readable medium. The readable medium may be a readable signal medium or a readable storage medium. The readable storage medium may be for example but not limited to, an electronic, magnetic, optical, electromagnetic, infrared, or semiconductor system, apparatus, or device, or any combination thereof. More specific examples of the readable storage medium (a non-exhaustive list) include: an electrical connection having one or more wires, a portable disk, a hard disk, a Random Access Memory (RAM), a Read-Only Memory (ROM), an Erasable Programmable Read-Only Memory (EPROM or flash memory), an optical fiber, a Compact Disk Read-Only Memory (CD-ROM), an optical storage device, a magnetic storage device, or any suitable combination thereof.

The computer-readable signal medium may include a data signal propagated in baseband or as a part of a carrier wave, which carries computer-readable program codes. Such a propagated data signal may take a variety of forms, including, but not limited to, an electromagnetic signal, an optical signal, or any suitable combination thereof. The readable signal medium may also be any readable medium other than a readable storage medium that may send, propagate, or transmit a program for use by or in connection with an instruction execution system, apparatus, or device.

Program codes included on a readable medium may be transmitted using any suitable medium, including but not limited to: wireless, electrical wires, optical cables, RFs, etc., or any suitable combination thereof.

Program codes for performing the operations of the present disclosure may be written in any combination of one or more programming languages, including an object oriented programming language such as Java, C++, etc., also including conventional procedural programming language—such as the “C” language or a similar programming language. The program codes can execute entirely on a user computing device, partially on the user device, as a stand-alone software package, partially on a remote computing device and partially on the user computing device, or entirely on the remote computing device or a server. In the case of the remote computing device, the remote computing device may be connected to the user computing device through any kind of network, including a Local Area Network (LAN) or Wide Area Network (WAN), or may be connected to an external computing device (for example, connected via the Internet through an Internet service provider).

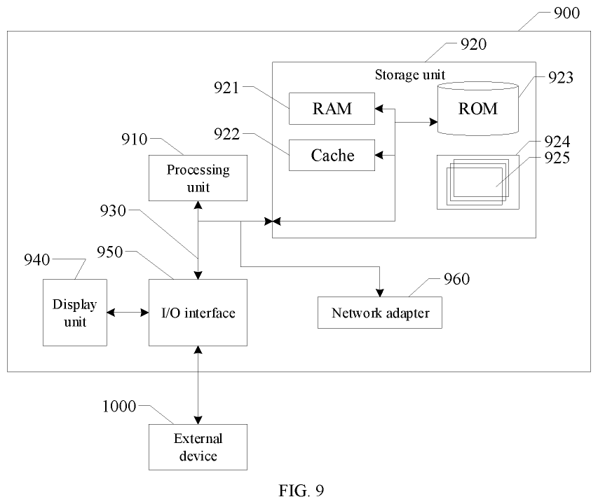

The exemplary embodiment of the present disclosure also provides an electronic device capable of implementing the above method. The electronic device 900 according to the exemplary embodiment of the present disclosure is described below with reference to FIG. 9. The electronic device 900 shown in FIG. 9 is merely an example, and should not impose any limitation on the functions and the scope of use of the embodiments of the present disclosure.

As shown in FIG. 9, the electronic device 900 may be represented in the form of a general-purpose computing device. Components of the electronic device 900 may include, but are not limited to, at least one processing unit 910, at least one storage unit 920, a bus 930 connecting different system components (including the storage unit 920 and the processing unit 910), and a display unit 940.

The storage unit 920 stores a program code, and the program code may be performed by the processing unit 910, so that the processing unit 910 performs the steps according to various exemplary embodiments of the present disclosure described above in this specification. For example, the processing unit 910 may perform any one or more of the method steps in FIGS. 2 to 7.

The storage unit 920 may include a readable medium in the form of a volatile storage unit, for example, a random access storage unit (RAM) 921 and/or a cache storage unit 922, and may further include a read-only storage unit (ROM) 923.

The storage unit 920 may further include a program/utility tool 924 having a set of (at least one) program modules 925. Such program modules 925 include, but are not limited to: an operating system, one or more applications, other program modules, and program data. Each of these examples or some combination may include an implementation of the network environment.

The bus 930 may be one or more of several types of bus structures, including a storage unit bus or a storage unit controller, a peripheral bus, a graphics acceleration port, a processing unit, or a local bus using any of a variety of bus structures.

The electronic device 900 may also communicate with one or more external devices 1000 (for example, a keyboard, a pointing device, a Bluetooth device, etc.), and may also communicate with one or more devices that enable the user to interact with the electronic device 900, and/or communicate with any device (for example, a router, a modem, etc.) that enables the electronic device 900 to communicate with one or more other computing devices. This communication may be performed through an input/output (I/O) interface 950. Moreover, the electronic device 900 may also communicate with one or more networks (for example, a Local Area Network (LAN), a Wide Area Network (WAN), and/or a public network, such as the Internet) through the network adapter 960. As shown in FIG. 9, the network adapter 960 may communicate with other modules of the electronic device 900 through the bus 930. It should be understood that, although not shown in the figure, other hardware and/or software modules may be used in conjunction with the electronic device 900, including, but not limited to: microcode, a device driver, a redundant processing unit, an external disk drive array, a RAID system, a tape drive, a data backup storage system and the like.

In some embodiments, the method for predicting the RNA-protein interaction described in the present disclosure may be performed by the processing unit 910 of the electronic device. In some embodiments, the candidate RNA sequence and the candidate protein sequence, the training dataset for training each model and the like may be input through the user interaction interface 950. For example, the candidate RNA sequence and the candidate protein sequence and the training dataset for training each model are input through a user interaction interface of the electronic device. In some embodiments, the prediction result of the interaction of the candidate RNA sequence and the candidate protein sequence may be output to the external device 1000 through the output interface 950 for viewing by the user.

It could be readily understood by those skilled in the art from the description of the above embodiments that the exemplary embodiments described herein may be implemented by software or by means of software in conjunction with necessary hardware. Thus, the technical solution according to the embodiments of the present disclosure may be embodied in the form of a software product which may be stored on a non-transitory storage medium (which may be a CD-ROM, a USB stick, a mobile hard disk, etc.) or on a network, and includes several instructions to enable a computing device (which may be a personal computer, a server, a terminal apparatus, or a network device, etc.) to perform a method according to the exemplary embodiments of the present disclosure.

In addition, the above accompanying figures are merely illustrative description of processes included in the method according to the exemplary embodiments of the present disclosure, rather than for the purpose of limitation. It is easy to understand that the processes shown in the accompanying figures do not indicate or limit time sequences of these processes. Furthermore, it is also easy to understand that these processes may be performed, for example, synchronously or asynchronously in a plurality of modules.

It should be noted that although several modules or units of the device for action execution are mentioned in the detailed description above, this division is not mandatory. In fact, according to the embodiments of the present disclosure, the features and functions of two or more modules or units described above may be embodied in one module or unit. Conversely, the features and functions of one module or unit described above may be further divided into a plurality of modules or units to be embodied.

It should be understood that the present disclosure is not limited to the precise structure that has been described above and shown in the accompanying figures, and that various modifications and changes may be made without departing from the scope thereof. The scope of the present disclosure is limited only by the appended claims.

1. A method for predicting an RNA-protein interaction, comprising:
acquiring a candidate RNA sequence and a candidate protein sequence;
encoding the candidate RNA sequence to obtain an RNA vector sequence;
encoding the candidate protein sequence to obtain a protein vector sequence;
constructing a matching feature matrix according to the RNA vector sequence and the protein vector sequence; and
performing feature extraction on the matching feature matrix, and determining, according to an extracted matching feature, an interaction between the candidate RNA sequence and the candidate protein sequence.

2. The method for predicting the RNA-protein interaction of claim 1, wherein the encoding the candidate RNA sequence to obtain an RNA vector sequence, comprising:
converting the candidate RNA sequence into N base k-mer subsequences; and
vectorizing each base k-mer subsequence of the N base k-mer subsequences to obtain the RNA vector sequence.

3. The method for predicting the RNA-protein interaction of claim 2, wherein the vectorizing each base k-mer subsequence of the N base k-mer subsequences to obtain the RNA vector sequence, comprising:
encoding each base k-mer subsequence of the N base k-mer subsequences to obtain first vectors of the N base k-mer subsequences, and constituting the RNA vector sequence by the first vectors of the N base k-mer subsequences.

4. The method for predicting the RNA-protein interaction of claim 2, wherein the vectorizing each base k-mer subsequence of the N base k-mer subsequences to obtain the RNA vector sequence, comprising:
encoding each base k-mer subsequence of the N base k-mer subsequences to obtain first vectors of the N base k-mer subsequences; and
inputting the first vectors of the N base k-mer subsequences sequentially into a pre-trained recurrent neural network, outputting N base k-mer vectors, and constituting the RNA vector sequence by the N base k-mer vectors.

5. The method for predicting the RNA-protein interaction of claim 2, wherein the vectorizing each base k-mer subsequence of the N base k-mer subsequences to obtain the RNA vector sequence, comprising:
encoding each base k-mer subsequence of the N base k-mer subsequences to obtain first vectors of the N base k-mer subsequences; and
performing operation on the first vectors of the N base k-mer subsequences by using a first mapping matrix to obtain second vectors of the N base k-mer subsequences, and constituting the RNA vector sequence by the second vectors of the N base k-mer subsequences.

6. The method for predicting the RNA-protein interaction of claim 2, wherein the vectorizing each base k-mer subsequence of the N base k-mer subsequences to obtain the RNA vector sequence, comprising:
encoding each base k-mer subsequence of the N base k-mer subsequences to obtain first vectors of the N base k-mer subsequences;
performing operation on the first vectors of the N base k-mer subsequences by using a first mapping matrix to obtain second vectors of the N base k-mer subsequences; and
inputting the second vectors of the N base k-mer subsequences sequentially into a pre-trained recurrent neural network, outputting N base k-mer vectors, and constituting the RNA vector sequence by the N base k-mer vectors.

7. The method for predicting the RNA-protein interaction of claim 1, wherein the encoding the candidate protein sequence to obtain a protein vector sequence, comprising:
converting the candidate protein sequence into M amino acid k-mer subsequences; and
vectorizing each amino acid k-mer subsequence of the M amino acid k-mer subsequences to obtain the protein vector sequence.

8. The method for predicting the RNA-protein interaction of claim 7, wherein the vectorizing each amino acid k-mer subsequence of the M amino acid k-mer subsequences to obtain the protein vector sequence, comprising:
encoding each amino acid k-mer subsequence of the M amino acid k-mer subsequences to obtain first vectors of the M amino acid k-mer subsequences, and constituting the protein vector sequence by the first vectors of the M amino acid k-mer subsequences.

9. The method for predicting the RNA-protein interaction of claim 7, wherein the vectorizing each amino acid k-mer subsequence of the M amino acid k-mer subsequences to obtain the protein vector sequence, comprising:
encoding each amino acid k-mer subsequence of the M amino acid k-mer subsequences to obtain first vectors of the M amino acid k-mer subsequences; and
inputting the first vectors of the M amino acid k-mer subsequences sequentially into a pre-trained recurrent neural network, outputting M amino acid k-mer vectors, and constituting the protein vector sequence by the M amino acid k-mer vectors.

10. The method for predicting the RNA-protein interaction of claim 7, wherein the vectorizing each amino acid k-mer subsequence of the M amino acid k-mer subsequences to obtain the protein vector sequence, comprising:
encoding each amino acid k-mer subsequence of the M amino acid k-mer subsequences to obtain first vectors of the M amino acid k-mer subsequences; and
performing operation on the first vectors of the M amino acid k-mer subsequences by using a second mapping matrix to obtain second vectors of the M amino acid k-mer subsequences, and constituting the protein vector sequence by the second vectors of the M amino acid k-mer subsequences.

11. The method for predicting the RNA-protein interaction of claim 7, wherein the vectorizing each amino acid k-mer subsequence of the M amino acid k-mer subsequences to obtain the protein vector sequence, comprising:
encoding each amino acid k-mer subsequence of the M amino acid k-mer subsequences to obtain first vectors of the M amino acid k-mer subsequences;
performing operation on the first vectors of the M amino acid k-mer subsequences by using a second mapping matrix to obtain second vectors of the M amino acid k-mer subsequences; and
inputting the second vectors of the M amino acid k-mer subsequences sequentially into a pre-trained recurrent neural network, outputting M amino acid k-mer vectors, and constituting the protein vector sequence by the M amino acid k-mer vectors.

12. The method for predicting the RNA-protein interaction of claim 1, wherein the constructing a matching feature matrix according to the RNA vector sequence and the protein vector sequence, comprising:
obtaining a calculated matching degree score by calculating a matching degree between a base k-mer vector in the RNA vector sequence and an amino acid k-mer vector in the protein vector sequence; and
constructing the matching feature matrix by taking the calculated matching degree score as an element of the matching feature matrix.

13. The method for predicting the RNA-protein interaction of claim 12, wherein the calculating a matching degree between a base k-mer vector in the RNA vector sequence and an amino acid k-mer vector in the protein vector sequence, comprising:
calculating the matching degree m(hᵢ^{R}, hⱼ^{P}) between the i-th base k-mer vector hᵢ^{R} in the RNA vector sequence and the j-th amino acid k-mer vector hⱼ^{P} in the protein vector sequence according to:
$$m ( {h}_{i}^{R} , {h}_{j}^{p} ) = exp ( - {‖ \frac{{h}_{i}^{R}}{\vert {h}_{i}^{R} \vert} - \frac{{h}_{j}^{p}}{\vert {h}_{j}^{p} \vert} ‖}_{2} ) ;$$
wherein |hᵢ^{R}| represents a length of hᵢ^{R}, and |hⱼ^{P}| represents a length of hⱼ^{P}.

14. The method for predicting the RNA-protein interaction of claim 1, wherein the constructing a matching feature matrix according to the RNA vector sequence and the protein vector sequence, comprising:
performing a dot-product operation on the RNA vector sequence and the protein vector sequence to obtain the matching feature matrix.

15. The method for predicting the RNA-protein interaction of claim 1, wherein the performing feature extraction on the matching feature matrix, comprising:
performing feature extraction on the matching feature matrix by using a feature extraction network to obtain an original feature; and
performing operation on the original feature by using a third mapping matrix to obtain the extracted matching feature.

16. (canceled)

17. The method for predicting the RNA-protein interaction of claim 1, wherein the determining, according to an extracted matching feature, an interaction between the candidate RNA sequence and the candidate protein sequence, comprising:
obtaining an interaction predicted value between the candidate RNA sequence and the candidate protein sequence according to the extracted matching feature; and
determining the interaction between the candidate RNA sequence and the candidate protein sequence according to the interaction predicted value, wherein the determining the interaction between the candidate RNA sequence and the candidate protein sequence according to the interaction predicted value, comprising:
determining, in response to the interaction predicted value meeting a preset threshold condition, that the interaction exists between the candidate RNA sequence and the candidate protein sequence.

18. The method for predicting the RNA-protein interaction of claim 17, wherein the obtaining an interaction predicted value between the candidate RNA sequence and the candidate protein sequence according to the extracted matching feature, comprising:
inputting the extracted matching feature into a classifier, and outputting a probability of the presence of the interaction between the candidate RNA sequence and the candidate protein sequence.

19. The method for predicting the RNA-protein interaction of claim 18, wherein the probability of the presence of the interaction between the candidate RNA sequence and the candidate protein sequence is
$$P ( 1 \vert r , p ) = \frac{{e}^{{c}_{1}}}{{e}^{{c}_{1}} + {e}^{{c}_{0}}} ;$$
wherein r represents the candidate RNA sequence, p represents the candidate protein sequence, c₀ represents a first feature value in the extracted matching feature, and c₁ represents a second feature value in the extracted matching feature.

20. (canceled)

21-26. (canceled)

27. A non-transitory computer-readable storage medium, having a computer program stored thereon, wherein the computer program, when performed by a processor, causes the processor to:
acquire a candidate RNA sequence and a candidate protein sequence;
encode the candidate RNA sequence to obtain an RNA vector sequence;
encode the candidate protein sequence to obtain a protein vector sequence;
construct a matching feature matrix according to the RNA vector sequence and the protein vector sequence; and
perform feature extraction on the matching feature matrix, and determining, according to an extracted matching feature, an interaction between the candidate RNA sequence and the candidate protein sequence.

28. An electronic device, comprising:
a processor; and
a memory for storing executable instructions for the processor;
wherein the processor is configured to perform following acts by executing the executable instructions:
acquiring a candidate RNA sequence and a candidate protein sequence;
encoding the candidate RNA sequence to obtain an RNA vector sequence;
encoding the candidate protein sequence to obtain a protein vector sequence;
constructing a matching feature matrix according to the RNA vector sequence and the protein vector sequence; and
performing feature extraction on the matching feature matrix, and determining, according to an extracted matching feature, an interaction between the candidate RNA sequence and the candidate protein sequence.
